(19) United States
(12) Patent Application Publication
(10) Pub. No.: US 2014/0024596 A1
(43) Pub. Date: Jan. 23, 2014

(54) DISEASE INHIBITING AGENT

(71) Applicant: NITTA GELATIN INC., Osaka (JP)

(72) Inventors: Fumihito SUGIHARA, Osaka (JP); Naoki INOUE, Osaka (JP); Hiroshi MANO, Saitama (JP)

(21) Appl. No.: 13/915,206

(22) Filed: Jun. 11, 2013

Related U.S. Application Data
(63) Continuation of application No. PCT/JP2011/078645, filed on Dec. 12, 2011.

(30) Foreign Application Priority Data
Dec. 14, 2010 (JP) 2010-277932
Jan. 14, 2011 (JP) 2011-006035
Jul. 1, 2011 (JP) PCT/JP2011/065186

(51) Int. Cl. C07K 7/08; A61K 31/713; A61K 38/08; C07K 7/06; A61K 38/10

(52) U.S. Cl. CPC C07K 7/08; C07K 7/06; A61K 38/10; A61K 38/08; A61K 31/713
USPC 514/16.8; 530/327; 530/328; 530/329; 514/21.5; 514/21.6; 514/21.7; 514/21.8; 514/16.9

(57) ABSTRACT

At least one peptide molecule selected from EGDGHLGKPGROGE (SEQ ID NO:1), EKDGHPGKPGROGE (SEQ ID NO:2), G(POG)₄, (POG)₃, G(POG)₂, (POG)₂, (POG)₄, (POG)₅ and G(POG)₃, and pharmaceutically acceptable salts thereof is effective for inhibiting various diseases such as osteoporosis, osteoarthritis and pressure ulcer. The peptide molecule is easily absorbed into a body and migrates into cells in an intestinal tract, and strongly binds to a nucleic acid compound or the like to form a complex, and thus functions well as a carrier component for delivering the nucleic acid compound or the like without causing the problems associated with conventional DDS techniques.

### CROSS REFERENCE TO RELATED APPLICATIONS

The present application is a continuation of International Application No. PCT/JP2011/078645 filed Dec. 12, 2011, which claims priorities to Japanese Patent Application No. 2010-277932 filed Dec. 14, 2010, Japanese Patent Application No. 2011-006035 filed Jan. 14, 2011 and International Application No. PCT/JP2011/065186 filed Jul. 1, 2011, the entire contents of each of these applications being incorporated herein by reference in their entirety.

### TECHNICAL FIELD

The present invention relates to a disease inhibiting agent. More specifically, the present invention relates to a disease inhibiting agent comprising a peptide molecule having a specific structure, and functioning as an active ingredient for inhibiting osteoporosis, osteoarthritis, pressure ulcer and so on, the disease inhibiting agent being also used as a carrier component effective for inhibiting various diseases by a nucleic acid compound such as miRNA or siRNA as an active ingredient. In the present invention, the term “inhibition” is used in the both meanings of “prevention” that inhibits onset of a symptom, and “treatment” that suppresses a developed symptom.

### BACKGROUND ART

Osteoporosis is a condition that is associated with reduction in absolute bone quantity but not with qualitative change in bone. Bone is resorbed and formed consistently, and when the difference arises between the resorption rate and the formation rate, and the formation of bone is in a negative equilibrium, osteoporosis will occur. The resorption of bone is assumed by osteoclast, and the more significant the differentiation and activation of osteoclast are, the higher the bone resorption rate is. On the other hand, the bone formation is assumed by osteoblast, and the more significant the differentiation and activation of osteoblast are, the higher the bone formation rate is.

Osteoarthritis is such a disease that chronic degenerative change and proliferative change concurrently occur in a joint, and the form of the joint changes. Articular cartilage is gradually abraded or lost, and bone will become exposed. Since articular cartilage lacks a vascular system, the repair and regeneration of joint sliding part chondrocytes and costal cartilage tissues are particularly difficult in comparison with those in bone tissues having blood vessels. In particular, when bone tissues that support articular cartilage are sparse (osteoporosis), the function of the joint part is interfered, and as a result osteoarthritis (osteoarthritis) is developed.

Pressure ulcer is such a condition that skin and soft tissues in the site where the bone protrudes get a circulatory disorder due to prolonged compression between the bone and the bed and become necrotic during prolonged bed rest.

As an efficacy of peptide on the symptoms as described above, an efficacy on osteoarthritis has been reported, and there are known, for example, a joint-reinforcing beverage comprising collagen peptide and a glucosamine salt as active ingredients at pH 2 to 5 (Japanese Laid-Open Patent Publication No. 2002-125638: Patent Literature 1), an ameliorating agent for chronic rheumatism or osteoarthritis comprising tripeptide having an amino acid sequence of Gly-X-Y obtained by decomposing a collagen ingredient or a gelatin ingredient by a collagenase enzyme (Japanese Laid-Open Patent Publication No. 2002-255847: Patent Literature 2), and an oral arthropathy therapeutic agent or functional food comprising at least one selected from collagen and collagen peptide, and at least one selected from amino sugar, mucopolysaccharides, and uronic acid (Japanese Laid-Open Patent Publication No. 2003-48850: Patent Literature 3).

However, the aforementioned conventional techniques merely show that collagen, collagen peptide that is a mixture of various peptide molecules, or specific tripeptide is effective for prevention or treatment of osteoarthritis, and a peptide structure that is effective for prevention or treatment of diseases in the broad sense including osteoporosis, pressure ulcer and so on as well as osteoarthritis, is not clarified.

In recent years, RNA medicine using a nucleic acid compound such as miRNA (micro RNA) or siRNA (small interfering RNA) attracts attention.

However, in RNA medicine, a drug delivery system (DDS) for making the medicine selectively act on its target in a body has not been satisfactorily established, and in particular, there is still no effective oral administration type delivering carrier. RNA medicine faces not only the problem that normal cells and tissues other than the target are damaged, but also the problem that RNA medicine should be administered in a larger amount than required because of its poor delivery efficiency, and hence improvement in drug delivery system (DDS) in the meaning of solving these problems is demanded.

For solving the aforementioned problems, a large number of DDS techniques have been proposed, however, an effective oral administration type delivering carrier and a DDS technique having sufficient applicability are not known. The conventional DDS techniques and problems associated with these are as follows.

There are known a technique for producing an anionic drug-encapsulated nanoparticle including: the step of forming a nanoparticle in which a mixed liquid of a solution of at least a solution of an anionic drug (nucleic acid compound or the like) and a solution dissolving a biocompatible polymer in an organic solvent is added to an aqueous solution dissolving polyvinyl alcohol and a cationic polymer, to generate a suspension of an anionic drug encapsulated nanoparticle in which the anionic drug is encapsulated in the biocompatible polymer, the step of distilling off the organic solvent from the suspension of the anionic drug encapsulated nanoparticle, and the step of further encapsulating an anionic drug in an outer layer of the anionic drug encapsulated nanoparticle (Japanese Laid-Open Patent Publication No. 2007-99631: Patent Literature 4); a siRNA-hydrophilic polymer conjugate formed of a hydrophilic polymer and siRNA that are bound covalently (Japanese National Patent Publication No. 2009-504179: Patent Literature 5); a spherical drug delivery system based on a polymer carrier wherein at least one signal substance for transportation through a biological barrier, and at least one active substance are stored, and the carrier, the signal substance and the active substance do not mutually have a covalent bond (Japanese National Patent Publication No. 2009-512722: Patent Literature 6); a method of using a hemagglutination active protein derived from Clostridium bacteria as an intracellular introduction carrier of nucleic acid (Japanese Laid-Open Patent Publication No. 2009-81997: Patent Literature 7) and so on, however, no satisfactory effect has been obtained when they were orally administered because they were not intestinally absorbed, and migration of the carrier itself to the target cell was insufficient even when they were topically administered because the carrier did not readily migrate into a target cell. Further, the binding of the carrier and a nucleic acid compound as an active ingredient was insufficient, and the function of the carrier as a carrier was also insufficient. As a result, there is a problem that a nucleic acid compound cannot be delivered into a specific target cell efficiently.

### CITATION LIST

### Patent Literature

PTL 1: Japanese Laid-Open Patent Publication No. 2002-125638
PTL 2: Japanese Laid-Open Patent Publication No. 2002-255847
PTL 3: Japanese Laid-Open Patent Publication No. 2003-48850
PTL 4: Japanese Laid-Open Patent Publication No. 2007-99631
PTL 5: Japanese National Patent Publication No. 2009-504179
PTL 6: Japanese National Patent Publication No. 2009-512722
PTL 7: Japanese Laid-Open Patent Publication No. 2009-81997

### SUMMARY OF INVENTION

### Technical Problem

In light of the above, the problem to be solved by the present invention is to search for the entity of a peptide molecule effective for inhibiting various diseases such as osteoporosis, osteoarthritis and pressure ulcer, in particular, a novel substance having characteristics of being easily absorbed in a body and migrating into cells in an intestinal tract, and strongly binding to a nucleic acid compound electrostatically to form a complex in addition to the easiness of absorption into a body and the migration into cells, and hence having excellent bindability with other active ingredient, and thus capable of holding the other active ingredient surely and delivering the other active ingredient to a diseased site due to the excellent migratability, and thus capable of exerting a function as a carrier component for delivering other active ingredient such as a nucleic acid compound into a target cell without causing the aforementioned problem associated with the conventional DDS techniques, and to provide a disease inhibiting agent comprising such a component.

### Solution to Problem

The present inventors have made diligent efforts for solving the problem as described above. In that process, while we have already confirmed that Hyp-Gly and Pro-Gly are effective for inhibiting a disease and applied for a patent (Japanese Laid-Open Patent Publication No. 2010-106003), we have also examined the efficacy of other peptide molecules.

As a result of the aforementioned examination, the present inventors have found that a peptide molecule having a specific structure found by ourselves is easily absorbed into a body in an intestinal tract, and functions well as an active ingredient of a disease inhibiting agent, and is able to solve the aforementioned problem associated with the conventional DDS techniques because the peptide molecule having a specific structure has excellent performance as a carrier component of RNA medicine.

Concretely, we have found that, the peptide molecule inhibits differentiation and activation of osteoclast, increases differentiation and activation of osteoblast, and inhibits degeneration of chondrocyte, for example, thereby modulating differentiation thereof, and that it is effective for inhibiting osteoporosis and osteoarthritis, and have found that the peptide molecule also recovers the amount of tropocollagen in skin dermis and inhibits pressure ulcer.

We have also found that the peptide molecule having a specific structure is excellent in biocompatibility because it has a specific structure derived from an organism, and easily migrates through an intestinal tract into a body, and further into cells, so that it is very effective as a disease inhibiting agent of oral administration type.

Further, since the peptide molecule having a specific structure binds well to an anionic nucleic acid compound electrostatically and is difficult to be cut during transportation when it is cationized by being dipped in an acidic aqueous solution, we have also found that the peptide molecule functions not only as an active ingredient by itself, but also functions well as a carrier component for delivering a nucleic acid compound such as miRNA or siRNA into a target cell as an active ingredient. As a result, a nucleic acid compound can be transferred into a target cell with a small amount and high efficiency. Such an excellent function is not exerted by a dipeptide such as Hyp-Gly or Pro-Gly, for example, and this would be because in contrast to the peptide molecule having a specific structure found by the present inventors, which is an oligopeptide composed of six or more bound amino acids, a dipeptide is composed of two bound amino acids, and has less sites derived from amino acid which bind with an anionic nucleic acid compound, so that sufficient electrostatic binding force is not generated.

In the case of limiting a tumor cell as a target, by allowing the peptide molecule having a specific structure to form a complex by an electrostatic bond in blood by a co-administration method where the peptide molecule is orally administered and the nucleic acid compound is topically administered, rather than administering the peptide molecule and the nucleic acid compound that have been electrostatically bound, it is possible to deliver the nucleic acid compound into a target tumor cell with a small amount and high efficiency. The DDS techniques by such co-administration have not been available by conventional DDS carriers as described in the foregoing Patent Literatures 4 to 7, namely by conventional DDS carriers that are not intestinally absorbed, and will not migrate into blood.

Confirming these facts, we have accomplished the present invention.

That is, a disease inhibiting agent according to the present invention comprises as an essential ingredient at least one peptide molecule selected from the group consisting of Glu-Gly-Asp-Gly-His-Leu-Gly-Lys-Pro-Gly-Arg-Hyp-Gly-Glu (SEQ ID NO:1), Glu-Lys-Asp-Gly-His-Pro-Gly-Lys-Pro-Gly-Arg-Hyp-Gly-Glu (SEQ ID NO:2), Gly-(Pro-Hyp-Gly)₄ (SEQ ID NO:3), (Pro-Hyp-Gly)₃ (SEQ ID NO:4), Gly-(Pro-Hyp-Gly)₂ (SEQ ID NO:5), (Pro-Hyp-Gly)₂ (SEQ ID NO:6), (Pro-Hyp-Gly)₄ (SEQ ID NO:7), (Pro-Hyp-Gly)₅ (SEQ ID NO:8) and Gly-(Pro-Hyp-Gly)₃ (SEQ ID NO:9), and pharmaceutically acceptable salts thereof. Further, the present invention provides a novel substance, Glu-Gly-Asp-Gly-His-Leu-Gly-Lys-Pro-Gly-Arg-Hyp-Gly-Glu (SEQ ID NO:1), Glu-Lys-Asp-Gly-His-Pro-Gly-Lys-Pro-Gly-Arg-Hyp-Gly-Glu (SEQ ID NO:2), Gly-(Pro-Hyp-Gly)₄ (SEQ ID NO:3), (Pro-Hyp-Gly)₃ (SEQ ID NO:4), Gly-(Pro-Hyp-Gly)₂ (SEQ ID NO:5), (Pro-Hyp-Gly)₂ (SEQ ID NO:6), (Pro-Hyp-Gly)₄ (SEQ ID NO:7) or a pharmaceutically acceptable salt thereof, or a mixture thereof.

In the following, for simplification, these peptide molecules are also referred to simply as “peptide molecule having a specific structure”. Further, the abbreviations (e.g., Pro) standing for respective amino acid units forming the peptide molecule are further abbreviated concretely in the way Pro=P, Hyp=O, Gly=G, Glu=E, Asp=D, His=H, Leu=L, Lys=K, Arg=R by one alphabetic character.

Therefore, the peptide molecule having a specific structure is at least one peptide molecule selected from the group consisting of EGDGHLGKPGROGE (SEQ ID NO:1), EKDGHPGKPGROGE (SEQ ID NO:2), G(POG)₄, (POG)₃, G(POG)₂, (POG)₂, (POG)₄, (POG)₅ and G(POG)₃, and pharmaceutically acceptable salts thereof, by the aforementioned abbreviations.

### Advantageous Effects of Invention

According to the present invention, it is possible to effectively suppress symptoms of osteoporosis, osteoarthritis, pressure ulcer and the like. In particular, since the peptide molecule having a specific structure serving as an active ingredient easily migrates into a body or into cells in an intestinal tract, it is also appropriate for oral administration.

Further, since the peptide molecule having a specific structure has a characteristic of strongly binding to a nucleic acid compound or the like to form a complex, not only the peptide molecule itself is used as an active ingredient, but also the peptide molecule can be made to function as a carrier component, to deliver, for example, a nucleic acid compound or the like as an active ingredient into a target cell very efficiently and allow it to act.

### DESCRIPTION OF EMBODIMENTS

In the following, the disease inhibiting agent of the present invention will be specifically described, however, the scope of the present invention is not limited to the description, and those not exemplified in the description may also be appropriately modified without departing from the scope of the present invention.

[Peptide Molecule Having a Specific Structure]

The disease inhibiting agent of the present invention comprises as an essential ingredient a peptide molecule having a specific structure, namely at least one peptide molecule selected from the group consisting of EGDGHLGKPGROGE (SEQ ID NO:1), EKDGHPGKPGROGE (SEQ ID NO:2), G(POG)₄, (POG)₃, G(POG)₂, (POG)₂, (POG)₄, (POG)₅ and G(POG)₃, and pharmaceutically acceptable salts thereof.

The pharmaceutically acceptable salts include, for example, inorganic acid salts such as hydrochloride, sulfate and phosphate, organic acid salts such as methanesulfonate, benzenesulfonate, succinate and oxalate, inorganic base salts such as sodium salt, potassium salt and calcium salt, and organic base salts such as triethylammonium salt.

In the peptide molecule having a specific structure, each amino acid unit may be chemically modified, and as to a hydroxyproline unit, a hydroxyl group may be chemically modified.

In the present invention, the “peptide molecule having a specific structure” includes those chemically modified and those not chemically modified. In the following, the peptide molecule having a specific structure may also be indicated only by abbreviations (for example, the “peptide molecule of (Pro-Hyp-Gly)₅ (SEQ ID NO:8)” is simply indicated by “(Pro-Hyp-Gly)₅” or “(POG)₅”).

When the peptide molecule having a specific structure is chemically modified, it is dissoluble in weak acidic to neutral condition, and improvement in compatibility with other active ingredient as will be described later is also expected. Concretely, examples include chemical modification such as O-acetylation for a hydroxyl group in a hydroxyproline residue, chemical modification such as esterification or amidation for an α-carboxyl group in a glycine residue, chemical modification such as polypeptidylation, succinylation, maleylation, acetylation, deamination, benzoylation, alkylsulfonylation, allylsulfonylation, dinitrophenylation, trinitropheylation, carbamylation, phenylcarbamylation or thiolation for an α-amino group in a proline residue. Appropriate chemical modification may be selected depending on the kind or the like of other active ingredient as will be described later.

Further, examples as cationization of the peptide molecule having a specific structure, include ethylenediamination, spermination and the like.

The peptide molecule having a specific structure may be prepared, for example, by treating collagen or gelatin with an enzyme in two steps or synthesized from amino acids as will be described later. Chemical modification includes a known means as will be described later. However, it may be prepared by a method other than these methods, and for example, a method of omitting a primary enzymatic treatment or a method of concurrently conducting a primary enzymatic treatment and a secondary enzymatic treatment in place of a two-step enzymatic treatment method as will be described later.

<Two-Step Enzymatic Treatment of Collagen or Gelatin>

Collagen peptide containing the peptide molecule having a specific structure can be prepared by a two-step enzymatic treatment that includes subjecting collagen or gelatin to a primary enzymatic treatment in a generally method, and to a secondary enzymatic treatment by an enzyme having aminopeptidase activity.

In the above, the “aminopeptidase activity” basically means a peptidase having a function of liberating amino acid from the N terminal of a peptide chain, and concretely includes for example “aminopeptidase P activity”, “aminopeptidase N activity” and the like. The “aminopeptidase P activity” acts when proline exists at the second position from the N terminal, and the “aminopeptidase N activity” acts when amino acid other than proline exists at the second position from the N terminal. In this way, they may be used differently depending on the situation, and any of these may be used.

Here, as the enzyme used in the secondary enzymatic treatment, an enzyme having the aforementioned aminopeptidase activity and another activity in addition may be used, or an enzyme having another activity may be used together with an enzyme having aminopeptidase activity, depending on the purpose such as decomposition of a byproduct, the kind of collagen that is a starting material, and the kind of the enzyme used in the primary enzymatic treatment.

As such activity other than the aminopeptidase activity, for example, dipeptidase activity such as prolidase activity or hydroxyprolidase activity may be allowed to act, to thereby decompose the byproduct dipeptide. Further, since the aminopeptidase activity basically liberates amino acid on the N terminal side one by one, decomposition in the primary enzymatic treatment may be insufficient depending on the kind of collagen that is a starting material or the kind of the enzyme used in the primary enzymatic treatment, and thus the time required for the secondary enzymatic treatment may be prolonged. For addressing this, for example, another activity such as an activity of prolyloligopeptidase that is endopeptidase that hydrolyzes the carboxyl group side of a proline residue may be allowed to act, to thereby cut and remove the unnecessary site as a lump of oligopeptide or the like. In this manner, it is possible to conduct the secondary enzymatic treatment more efficiently.

According to this two-step enzymatic treatment, by the primary enzymatic treatment, peptides having a relatively large molecular weight that are useful for reducing inflammation of bone and cartilage tissues via an oral immune tolerance mechanism are generated, and further by the secondary enzymatic treatment, peptides molecule having a specific structure are generated.

For example, by using aminopeptidase N, it is possible to liberate amino acids X₁, X₂ sequentially from the N terminal side in the structure of [X₁-X₂-Gly-Pro-Hyp-](X₁≠Pro and X₂≠Hyp), or to liberate the dipeptide Pro-Hyp when X₁=Pro, and X₂=Hyp in the structure. As a result, [(Gly-(Pro-Hyp-Gly)ₙ] (n=2 to 4) that is a peptide molecule having a specific structure is obtained.

Further, by aminopeptidase N, it is possible to cut a glycine-proline bond on the C terminal side in the structure of [(Pro-Hyp-Gly)ₙ-Pro-Hyp-Gly-Pro-Y-] (n=1 to 4, Y≠Hyp), and as a result, [(Pro-Hyp-Gly)ₙ₊₁](n=1 to 4) that is a peptide molecule having a specific structure is obtained. This is the finding first found by the present inventors.

Further, by aminopeptidase N, it is possible to liberate the part of “X₃-X₄-Gly” on the N terminal side in the structure of [X₃-X₄-Gly-(Pro-Hyp-Gly)₅](X₃≠Pro and X₄≠Hyp), and as a result, [(Pro-Hyp-Gly)ₙ] (n=5) that is a peptide molecule having a specific structure is obtained.

By aminopeptidase P, it is possible to liberate glycine on the N terminal in the structure of [Gly-(Pro-Hyp-Gly)ₙ](n=2 to 4), and as a result, [(Pro-Hyp-Gly)ₙ₊₁] (n=2 to 4) that is a peptide molecule having a specific structure is obtained. There is also the case that glycine on the N terminal does not liberate, and in such a case, [Gly-(Pro-Hyp-Gly)ₙ](n=2 to 4) that is a peptide molecule having a specific structure will partly remain.

The collagen include, but not limited to, for example, collagen derived from mammals such as cows or pigs, and collagen derived from fish such as shark or sea bream, and these may be obtained from bones or skin part of the mammals or from bones, skin or scale part of the fish. Concretely, the bones, skin, scale or the like may be subjected to a conventionally known treatment such as delipidation, decalcification or extraction.

The gelatin may be obtained by treating the collagen by a conventionally known method such as hot water extraction.

The enzyme used in the two-step enzymatic treatment of the collagen or gelatin is not particularly limited, but enzymes other than enzymes derived from pathogenic microorganisms are preferably used in consideration of the case that the obtained peptide molecule is used in food for specified health use.

As a treatment condition of the primary enzymatic treatment, for example, the treatment may be effected at 30 to 65° C. for 1 to 72 hours using 0.1 to 5 parts by weight of enzyme per 100 parts by weight of collagen or gelatin.

An average molecular weight of the collagen peptide obtained by the primary enzymatic treatment of the collagen or gelatin is preferably 500 to 2000, and more preferably 500 to 1800. The average molecular weight falling within the above range implies that peptides having a relatively large molecular weight are adequately generated.

While the enzyme may be inactivated as necessary after the primary enzymatic treatment, the inactivation temperature in this case is for example, 70 to 100° C.

As the enzyme used in the primary enzymatic treatment, any enzymes capable of cutting peptide bonds in collagen or gelatin may be used without particular limitation, however, an enzyme called proteolytic enzyme or protease is typically used. Concretely, examples include collagenase, thiol protease, serine protease, acidic protease, alkaline protease, and metal protease, which may be used singly or in combination of plural kinds. As the thiol protease, for example, plant derived proteases such as chymopapain, papain, bromelain and ficin, and animal derived proteases such as cathepsin and calcium-dependent protease are known. As the serine protease, trypsin, cathepsin D and so on are known, and as the acidic protease, pepsin, chymotrypsin and the like are known.

Further, in the secondary enzymatic treatment, an enzymatic reaction using, for example, an enzyme having aminopeptidase activity derived from Aspergillus as an enzyme is conducted. By this reaction, a peptide molecule having a specific structure not contained in the product of the primary enzymatic treatment is generated.

As a treatment condition of the secondary enzymatic treatment, for example, the treatment may be effected at 30 to 65° C. for 1 to 72 hours using 0.01 to 5 parts by weight of enzyme per 100 parts by weight of the product of the primary enzymatic treatment.

An average molecular weight of the collagen peptide obtained by the secondary enzymatic treatment is preferably 500 to 1800, and more preferably 500 to 1500. This secondary enzymatic treatment is principally intended to generate a peptide molecule having a specific structure, and it is preferred to conduct the secondary enzymatic treatment so that the average molecular weight falls within the aforementioned range for preventing relatively large peptides in the collagen peptide obtained by the primary enzymatic treatment from being excessively hydrolyzed.

It is necessary to inactivate the enzyme after the secondary enzymatic treatment, and the inactivation temperature is for example, 70 to 100° C.

Since the hydrolysate obtained by the two-step enzymatic treatment or the fermentation product obtained by the two-step enzymatic treatment and fermentation is a mixture containing amino acid and peptide components other than the peptide molecule having a specific structure, fractionation and purification may be conducted as necessary for obtaining the peptide molecule having a specific structure or a salt thereof. The method for fractionation and purification is not limited, and any conventionally known methods, for example, ultrafiltration, and various liquid chromatography methods such as gel filtration chromatography, ion exchange chromatography, reverse-phase chromatography, affinity chromatography and the like, and combination of these methods may be employed. Concretely, the fractionation and purification may be conducted, for example, in the following manner. In brief, first, about 2 g/10 mL of the hydrolysate or fermentation product is applied to an ion exchange column (e.g., DEAE TOYOPEARL 650M column (manufactured by TOSOH CORPORATION) or an SP TOYOPEARL 650M column (manufactured by TOSOH CORPORATION)) in two parts, and a void volume fraction eluted with distilled water is recovered. Then, the recovered fraction is applied to a column having an ion exchange group of the opposite polarity to that of the aforementioned ion exchange column (e.g., SP TOYOPEARL 650M column (manufactured by TOSOH CORPORATION) or a DEAE TOYOPEARL 650M column (manufactured by TOSOH CORPORATION)), and a void volume fraction eluted with distilled water is recovered. Then, this fraction is applied to a gel filtration column (e.g., Sephadex LH-20 column (manufactured by Pharmacia)), and eluted with an aqueous 30% methanol solution and the fraction corresponding to the position where a peptide molecule having a specific structure that is a chemical synthetic product elutes is recovered. This fraction is subjected to a high performance liquid chromatography (HPLC) loaded with a reverse-phase column (e.g., μBondasphere 5μC18 300 angstrom column (manufactured by Waters)), and fractionated by a straight concentration gradient of an aqueous acetonitrile solution of less than or equal to 32% containing 0.1% trifluoroacetic acid. Then, the recovered fraction of the peptide molecule having a specific structure is dried under reduced pressure to obtain the peptide molecule having a specific structure with high purity.

<Synthesis from Amino Acids>

The peptide molecule having a specific structure may be synthesized from amino acids.

As a method for synthesizing the peptide molecule having a specific structure, generally, (1) a solid-phase synthesis method and (2) a liquid-phase synthesis method (for example, see Japanese Laid-Open Patent Publication No. 2003-183298) are known, and in the case of the former method, methods of (A) Fmoc method and (B) Boc method are further known, and the peptide molecule having a specific structure may be synthesized in any method.

Detailed description will be made while taking a solid-phase method as one example.

It may be synthesized by a known solid-phase synthesis method wherein proline is immobilized to a carrier polystyrene, and a Fmoc group or a Boc group is used for protection of an amino group. That is, by a dehydration reaction using a bead of polystyrene polymer gel having a diameter of about 0.1 mm whose surface is modified with an amino group as a solid phase, and diisopropylcarbodiimide (DIC) as a condensing agent, hydroxyproline is bound to proline whose amino group is protected by a Fmoc (fluorenyl-methoxy-carbonyl) group, and the solid phase is washed well with a solvent, to remove the remaining hydroxyproline or the like. Thereafter, the protective group of the proline residue bound to the solid phase is removed (deprotected), and thus PO can be synthesized. Subsequently, in a similar manner, by making glycine bind to an amino group of a hydroxyproline residue of the PO (to form a peptide bond), POG can be obtained. In this way, by making amino acids bind sequentially, the intended peptide molecule can be synthesized.

<Chemical Modification>

The peptide molecule having a specific structure may be chemically modified. As a concrete means and treatment condition of chemical modification, a usual chemical modification technique for peptide is applied.

As to chemical modification of a hydroxyl group in a hydroxyproline residue, for example, O-acetylation may be achieved by treatment with acetic anhydride in an aqueous solvent or in a non-aqueous solvent.

As to chemical modification of an α-carboxyl group of a glycine residue, for example, esterification may be achieved by aerating a suspension in methanol with a dry hydrogen chloride gas, and amidation may be achieved by treatment with carbodiimide.

As other concrete examples of chemical modification, chemical modification techniques described in Patent Publication No. 62-44522, Patent Publication No. 5-79046 and so on may be applied.

[Disease Inhibiting Agent]

The disease inhibiting agent according to the present invention, includes preferably an osteoporosis inhibiting agent, an osteoarthritis inhibiting agent, a pressure ulcer inhibiting agent, and a complex of a nucleic acid compound and a peptide molecule (medicinal usage varies depending on the kind of the nucleic acid compound) and so on.

The disease inhibiting agent according to the present invention comprises as an essential ingredient the above peptide molecule having a specific structure and may comprise as an essential ingredient a peptide molecule having a specific structure contained in collagen peptide. Then, in this case, not only the mode that the disease inhibiting agent contains a peptide molecule having a specific structure chemically synthesized from amino acid or a peptide molecule having a specific structure isolated from collagen peptide that is hydrolysate of collagen or gelatin, but also the mode that the disease inhibiting agent may contain collagen peptide as it is and the peptide molecule having a specific structure is not isolated from the collagen peptide is allowable. Including the case of containing collagen peptide as it is, the disease inhibiting agent according to the present invention comprises as an essential ingredient the peptide molecule having a specific structure of the present invention, and the peptide molecule having a specific structure may be used in combination including the case that they are used in the form of collagen peptide.

The peptide molecule having a specific structure differs from amino acids and peptide molecules having a structure other than the peptide molecule having a specific structure (for example, G(POG)₅ in which Gly is further bound to (POG)₅ is not a peptide molecule having a specific structure). By containing the peptide molecule having a specific structure, excellent disease inhibiting effects (effect of suppressing symptoms such as osteoporosis, osteoarthritis and pressure ulcer, and effect of a carrier in RNA medicines) are expressed. These effects are concretely demonstrated in the performance evaluation test in the examples as will be described later.

<Use as an Active Ingredient>

First, description will be made for a disease inhibiting agent (e.g., osteoporosis inhibiting agent, osteoarthritis inhibiting agent, and pressure ulcer inhibiting agent) containing the peptide molecule having a specific structure as an active ingredient. When the peptide molecule is used as an active ingredient, it preferably contains at least one peptide molecule selected from the group consisting of EGDGHLGKPGROGE (SEQ ID NO:1), EKDGHPGKPGROGE (SEQ ID NO:2), G(POG)₄, (POG)₃, G(POG)₂, (POG)₂, (POG)₄, (POG)₅ and G(POG)₃, and pharmaceutically acceptable salts thereof.

The disease inhibiting agent containing the peptide molecule having a specific structure as an active ingredient may be administered orally or parenterally in various dosage forms. The dosage forms include, for example, liquids, tablets, granules, capsules, powders, injections, transdermal preparations, suppositories, nasal drops and inhalants, and preferably liquids that are directly administered to a diseased site, and tablets, granules and capsules that are orally administered. A dose of the peptide molecule having a specific structure varies depending on the condition or weight of the patient, the kind of the compound, the administration route and so on. In the case of direct administration to a diseased site, for example, the dose includes about 0.01 to 200 mg, preferably about 0.1 to 100 mg, and more preferably about 1 to 50 mg, per day for an adult. In the case of oral administration, for example, the dose includes about 0.1 to 1000 mg, preferably about 1 to 500 mg, and more preferably about 10 to 200 mg. In a preparation of another dosage form, the dose may be appropriately determined with reference to these administration amounts. These preparations may be administered daily in one to several divided doses, or may be administered once every one to several days.

In this case, it is preferred to blend the peptide molecule having a specific structure in a proportion of greater than or equal to 0.001 parts by weight with respect to the entire amount of the disease inhibiting agent according to the present invention. More preferably, it is blended in a proportion of greater than or equal to 0.01 parts by weight. There is a possibility that the effect of the present invention is not sufficiently expressed in a proportion of less than 0.001 parts by weight.

Further, when the disease inhibiting agent according to the present invention is directly injected into a diseased site, the content of the peptide molecule having a specific structure is preferably greater than or equal to 10 μmol/L.

The disease inhibiting agent according to the present invention may be a peptide molecule having a specific structure diluted with saline or the like, and can express the effect of the present invention sufficiently. However, other active ingredient and an ingredient for formulation may be added appropriately besides the peptide molecule having a specific structure unless the effect of the present invention is impaired.

The other active ingredient as described above includes glucosamine and/or its salt, chondroitin sulfate and the like, and these may be used singly or in combination of two or more kinds. Among these, glucosamine and/or its salt is preferred because it has a function of improving the disease inhibiting effect by the peptide molecule having a specific structure.

Further, the other active ingredient as described above includes a peptide molecule other than the peptide molecule having a specific structure or an amino acid may be added. For example, a peptide molecule having a relatively large molecular weight is useful for chronic rheumatoid arthritis or the like because it has an effect of alleviating inflammation of bone or cartilage tissues by the oral immune tolerance mechanism. For containing a peptide molecule other than the peptide molecule having a specific structure or an amino acid, after hydrolyzing collagen or gelatin to obtain collagen peptide containing the peptide molecule having a specific structure, the collagen peptide may be directly used while the peptide molecule having a specific structure is not isolated.

Further, as the other active ingredient as described above, calcium or glycosyl hesperidin may be used for the purpose of promoting the deposition of bone mineral, and vitamin C or the like may be used for the purpose of promoting the synthesis and deposition of collagen.

As a blending amount of the other active ingredient as described above, it is preferably used in a proportion of 0.001 to 20 parts by weight, and is more preferably used in a proportion of 0.01 to 20 parts by weight, with respect to the entire amount of the disease inhibiting agent. In particular, the blending amount of glucosamine and/or its salt is preferably 5 to 15 parts by weight with respect to the entire amount of the disease inhibiting agent. When it is less than 5 parts by weight, the effect of improving the effect of the peptide molecule having a specific structure may not be sufficiently exerted, and when it is more than 15 parts by weight, it may be discharged in urine or feces, and taken excessively.

As the ingredient for formulation, for example, an excipient such as crystalline cellulose may be used, and an appropriate amount may be selected depending on the dosage form thereof or the like.

As the dosage form of the disease inhibiting agent according to the present invention, for example, an oral administration form, and a direct injection form to a diseased site are recited. Since the peptide molecule having a specific structure is immediately absorbed in an intestinal tract and is little decomposed into amino acid, it is preferably taken orally.

In the case of oral administration, a mixture containing the peptide molecule having a specific structure and another active ingredient and an ingredient for formulation as described above may be prepared into tablets by tablet compression molding, or prepared into any other forms such as solid preparations including granules, powders, capsules or the like, liquid preparations including solutions, suspensions, emulsions or the like, and lyophilized preparations.

In the case of direct injection into a diseased site, the peptide molecule having a specific structure diluted with saline or the like is used, and other active ingredient as described above may be further used as necessary, preferably in such a concentration that the content of the peptide molecule having a specific structure is greater than or equal to 0.1 mol/L as described above.

<Use as a Carrier Component>

Next, description will be made for a disease inhibiting agent containing the peptide molecule having a specific structure as a carrier component to form an electrostatic complex with a nucleic acid compound.

While the peptide molecule having a specific structure functions as an active ingredient by itself as described above, it may be allowed to function as a carrier component for delivering a nucleic acid compound into the interior of a target cell utilizing intestinal absorptivity, ease of migration into a cell, and strong electrostatic bindability with a nucleic acid compound. In this case, since the nucleic acid compound functions as an active ingredient for inhibiting a disease, the role of the peptide molecule is different from that when the peptide molecule itself functions as an active ingredient.

The nucleic acid compound include, for example, miRNA and siRNA. More concretely, included are, for example, a gene expression cassette into which a gene encoding a substance such as infection-protective antigen in microorganism infection, biologically active substance, enzyme inhibiting substance, receptor inhibiting substance, oncogenic suppressing substance, apoptosis promoting substance, apoptosis suppressing substance, cell regeneration promoting substance, immunoreaction promoting substance, immunoreaction suppressing substance or the like is incorporated; ribozyme or antisense gene; nucleic acid having a function of suppressive ribonucleic acid, and the like. Here, the gene expression cassette refers to an expression vector that is appropriately constructed for expression of an exogenous gene in a cell.

As a method for forming an electrostatic complex of the peptide molecule having a specific structure and the nucleic acid compound, as the disease inhibiting agent according to the present invention, for example, the peptide molecule having a specific structure and the nucleic acid mixture may be mixed in a buffer. The buffer is not particularly limited, and may be appropriately selected from one that will not adversely affect on a cell or living body, for example, saline, phosphoric acid buffer, phosphate buffer, citrate buffer and so on.

The mixing ratio between the peptide molecule having a specific structure and the nucleic acid compound may be, for example, about 1:1 to 10:1, preferably about 1.1:1 to 5:1, and more preferably about 1.2:1 to 3:1 although it varies depending on the specific peptide and the specific nucleic acid compound, or on their affinity.

The pH of the buffer is not particularly limited, and is, for example, preferably in the range of pH 6.0 to 8.5, and more preferably in the range of pH 7.0 to 8.0.

The salt concentration is preferably 0 to 10%, and more preferably 0.7 to 1.1%. The salt includes sodium chloride, potassium chloride, magnesium chloride and the like, and among these, sodium chloride is preferred.

The electrostatic complex of the peptide molecule having a specific structure and the nucleic acid compound may be administered orally or parenterally in preparation of various dosage forms. The dosage forms include, for example, liquids, tablets, granules, capsules, powders, injections, transdermal preparations, suppositories, nasal drops, and inhalants, and preferably include liquids that are directly administered to a diseased site, and tablets, granules and capsules that are orally administered. A dose of the peptide molecule having a specific structure may be determined with reference to a dose of the corresponding nucleic acid compound although it varies depending on the kind of the nucleic acid compound, the condition or weight of the patient, the kind of the compound, the administration route and so on.

Further, the disease inhibiting agent of the present invention also effectively function in the mode of co-administration, namely in such a mode that the peptide molecule having a specific structure is orally administered, and the nucleic acid compound is topically administered. This owes that the peptide molecule having a specific structure having migrated into blood by oral administration associates with the topically administered nucleic acid compound in blood to form a complex (electrostatic complex of these), enabling expression of the function of the nucleic acid compound by incorporation into a target cell (for example, cancer cell). As a result, it is possible to introduce into a tumor cell efficiently without necessity of binding it electrostatically with the miRNA or siRNA in advance.

### EXAMPLES

In the following, the present invention will be described more concretely by way of performance evaluation tests for a peptide molecule of the essential ingredient of the disease inhibiting agent according to the present invention, and collagen peptide containing the same, and blending examples of the disease inhibiting agent, however, it is to be understood that the present invention will not be limited to these.

In the following context, “part(s) by weight” may also be indicated simply by “part(s)” and “% by weight” may also be indicated by “%” for simplification.

[Preparation of Peptide Molecule Having a Specific Structure]

As the peptide molecules having a specific structure for use in the performance evaluation tests and in the disease inhibiting agent as will be described later, the followings were used.

Specifically, (POG)₅ was obtained from PEPTIDE INSTITUTE INC., and EGDGHLGKPGROGE (SEQ ID NO:1) and EKDGHPGKPGROGE (SEQ ID NO:2), G(POG)₄, (POG)₄, G(POG)₃, (POG)₃, and G(POG)₂ and (POG)₂ were respectively obtained from PH Japan.

[Preparation of Other Peptide Molecule]

Other peptide molecule for comparison used in the performance evaluation test or in the disease inhibiting agent as will be described later were synthesized by a solid-phase method as described above.

In brief, first, using a bead of polystyrene polymer gel having a diameter of about 0.1 mm whose surface was modified with an amino group as a solid phase, 45 parts of glycine was allowed to bind to 45 parts of hydroxyproline whose amino group was protected with a Fmoc (fluorenyl-methoxy-carbonyl) group by dehydration reaction using 10 parts of diisopropyl carbodiimide (DIC) as a condensing agent (to form a peptide bond). Then the solid phase was washed well with a solvent (ethylalcohol) to remove the remaining hydroxyproline or the like. Then, by removing (deprotecting) the protective group in the hydroxyproline residue bound to the solid phase by infusion in trifluoroacetic acid, OG was synthesized.

For synthesis of each peptide molecule, a Liberty peptide synthesis system (manufactured by CEM Corporation) was used.

Also, PO, Ala-Hyp, Leu-Hyp, Phe-Hyp, Ser-Hyp, and POG were synthesized in a similar manner.

[Preparation of Collagen Peptide Containing Peptide Molecule Having a Specific Structure, 1]

Collagen peptide derived from pig skin (PC) containing a peptide molecule having a specific structure for use in the performance evaluation tests and in the disease inhibiting agent as will be described later was obtained according to the following method.

In brief, 1 kg of gelatin being a thermal-denatured product of collagen derived from pig skin (Type I collagen) was dissolved in 4 L of hot water at 75° C., and the temperature was adjusted to 60° C., and then as a primary reaction, 10 g of protease derived from yellow aspergillus was added and the reaction was retained at pH 5.0 to 6.0 at a temperature of 45 to 55° C. for 120 minutes for enzymatic hydrolysis treatment. Then, as a secondary enzymatic reaction, an Aspergillus oryzae extraction enzyme having aminopeptidase P activity was added at a final concentration of 1.5% to make the resultant soluble, and the reaction was allowed at 50° C. for 2 hours. After the reaction, the reaction liquid was heated at 100° C. for 10 minutes, and then cooled to 60° C., and filtered by using activated charcoal and a filtration aid (diatomaceous earth), and the obtained mother liquor was subjected to a high temperature sterilization treatment at 120° C. for 3 seconds. Then, the sterilized mother liquor was spray-dried to obtain collagen peptide derived from pig skin (PC).

The PC was subjected to thin-layer chromatography (TLC). In brief, after dropping 10 μg of PC solubilized in water on a TLC plate (product name “Cellulose F”, manufactured by Merck KGaA) (spot origin), and drying the same, the chromatogram was developed by a solvent (n-butanol:acetic acid:water=4:1:2). By spraying an isatin-Zn color forming liquid, and confirming correspondence of a coloring Rf value of a blue spot with an Rf value of each of the synthetic peptide molecules (POG)₅, (POG)₄, (POG)₃, and (POG)₂ on the same plate, it was confirmed that each peptide molecule in the PC was contained.

For the PC, MALDI-TOF/MS analysis was further conducted. Since the PC contained various kinds of peptide molecules and was difficult to be analyzed, the sample was fractionated by reverse-phase chromatography using a Sep-PakC18 cartridge column (manufactured by Waters), followed by lyophilization, and the sample was dissolved in 20 μL of MQ water, and subjected to the MALDI-TOF/MS analysis.

Concretely, in the MALDI-TOF/MS analysis, the mass was determined by combination of the Matrix assisted laser desorption ionization (MALDI) method and the Time of flight/mass (TOF/MS) method. As a matrix for MALDI, a supernatant of a solution of 0.1% TFA-containing 50% acetonitrile to which was added a trace amount of α-cyano-4-hydroxycinnamic acid (CHCA) was used. This solution was mixed with an equivalent amount of a sample to be analyzed, to prepare crystals. By irradiation with laser for a short time, the sample to be analyzed was ionized. Every mass spectrum was obtained by an Autoflex TOF/TOF mass spectrometer (manufactured by Bruker Daltonics) equipped with 337 nm nitrogen laser and accelerating ion at 6 kV. The obtained molecular peaks and ion peaks by CID-LIFT were analyzed.

From the analysis, it was confirmed that this PC also contains peptide molecules EGDGHLGKPGROGE (SEQ ID NO:1), EKDGHPGKPGROGE (SEQ ID NO:2), G(POG)₄, G(POG)₃, and G(POG)₂.

From the ion peak analysis in CID-LIFT of MALDI-TOF/MS, it was revealed that the PC contains 0.01% of EGDGHLGKPGROGE (SEQ ID NO:1), 0.01% of EKDGHPGKPGROGE (SEQ ID NO:2), 0.01% of (POG)₅, 0.02% of G(POG)₄, 0.1% of (POG)₄, 0.2% of G(POG)₃, 1% of (POG)₃, 2% of G(POG)₂, and 5% of (POG)₂.

In the ion peak analysis, m/z of EGDGHLGKPGROGE (SEQ ID NO:1) is 1421.639, m/z of EKDGHPGKPGROGE (SEQ ID NO:2) is 1476.706, m/z of (POG)₅ is 1354.6, m/z of G(POG)₄ is 1087.5, m/z of (POG)₄ is 1144.5, m/z of G(POG)₃ is 877.4, m/z of (POG)₃ is 820.5, m/z of G(POG)₂ is 610.3, and m/z of (POG)₂ is 553.4.

[Preparation of Collagen Peptide Containing Peptide Molecule Having a Specific Structure, 2]

Collagen peptide derived from fish scale (FC) containing a peptide molecule having a specific structure for use in the performance evaluation tests and in the disease inhibiting agent as will be described later was produced in similar operations as those in the production of PC except that gelatin derived from fish scale was used.

The FC was analyzed by TLC similarly to the case of PC, and the presence of peptide molecules (POG)₅, (POG)₄, (POG)₃, and (POG)₂ was confirmed.

Further, from the MALDI-TOF/MS analysis, it was confirmed that this FC also contains peptide molecules G(POG)₄, G(POG)₃ and G(POG)₂.

From the ion peak analysis in CID-LIFT of MALDI-TOF/MS, it was revealed that the FC contains 0.01% of (POG)₅, 0.02% of G(POG)₄, 0.1% of (POG)₄, 0.2% of G(POG)₃, 1% of (POG)₃, 2% of G(POG)₂, and 5% of (POG)₂.

[Preparation of Collagen Peptide Containing Peptide Molecule Having a Specific Structure, 3]

Collagen peptide derived from pig skin (PC-CP) containing a peptide molecule having a specific structure for use in the performance evaluation tests and in the disease inhibiting agent as will be described later was obtained according to the following method.

In brief, 1 kg of gelatin being a thermal-denatured product of collagen derived from pig skin (Type I collagen) was dissolved in 4 L of 20 mM Tris-HCl buffer (pH 7.5) under warming, and then cooled to 40° C., and then as a primary enzymatic reaction, 1 g of collagenase (produced by Nitta Gelatin Inc., Collagenase N2) was added and the reaction was retained at pH 7.0 to 7.8 at a temperature of 40° C. for 18 hours for an enzymatic decomposition treatment. Then, as a secondary enzymatic reaction, an Aspergillus niger extraction enzyme having both aminopeptidase P and prolyloligopeptidase activities was added at a final concentration of 1.0% to the reaction liquid, and solubilized, and then allowed to react at pH 4.0, at 50° C. for 2 hours. After the reaction, the reaction liquid was heated at 100° C. for 10 minutes, and then cooled to 60° C., and filtered by using activated charcoal and a filtration aid (diatomaceous earth), and the obtained mother liquor was subjected to a high temperature sterilization treatment at 120° C. for 3 seconds. Then, the sterilized mother liquor was spray-dried to obtain PC-CP.

Here, by cutting and removing the unnecessary site on the N terminal side as a lump by using the enzyme having prolyloligopeptidase activity as well as the enzyme used in the secondary enzymatic reaction, the peptide molecule having a specific structure can be obtained efficiently.

The PC-CP was analyzed by TLC in a similar manner to the case of the PC, and the presence of peptide molecules EGDGHLGKPGROGE (SEQ ID NO:1), EKDGHPGKPGROGE (SEQ ID NO:2), (POG)₅, (POG)₄, (POG)₃, and (POG)₂ was confirmed.

Further, from the MALDI-TOF/MS analysis, it was confirmed that this PC-CP also contains peptide molecules G(POG)₄, G(POG)₃ and G(POG)₂.

From the ion peak analysis in CID-LIFT of MALDI-TOF/MS, it was revealed that the PC-CP contains 0.01% of EGDGHLGKPGROGE (SEQ ID NO:1), 0.01% of EKDGHPGKPGROGE (SEQ ID NO:2), 0.02% of (POG)₅, 0.04% of G(POG)₄, 0.2% of (POG)₄, 0.4% of G(POG)₃, 4% of (POG)₃, and 10% of (POG)₂.

[Preparation of Collagen Peptide Containing Peptide Molecule Having a Specific structure, 4]

Collagen peptide derived from pig skin (PC-2) containing a peptide molecule having a specific structure for use in the performance evaluation tests and in the disease inhibiting agent as will be described later was obtained in similar operations as those in the production of PC except that an Aspergillus oryzae extraction enzyme having aminopeptidase N activity was used in the secondary enzymatic reaction.

The PC-2 was analyzed by TLC similarly to the case of PC, and the presence of peptide molecules (POG)₅, (POG)₄, (POG)₃, and (POG)₂ was confirmed.

Further, from the MALDI-TOF/MS analysis, it was confirmed that this PC-2 also contains peptide molecules G(POG)₄, G(POG)₃, and G(POG)₂.

From the ion peak analysis in CID-LIFT of MALDI-TOF/MS, it was revealed that the PC-2 contains 0.01% of (POG)₅, 0.03% of G(POG)₄, 0.1% of (POG)₄, 0.3% of G(POG)₃, 1% of (POG)₃, 3% of G(POG)₂, and 4% of (POG)₂.

[Preparation of Collagen Peptide Containing Peptide Molecule Having a Specific Structure, 5]

Collagen peptide derived from fish scale (FC-2) containing a peptide molecule having a specific structure for use in the performance evaluation tests and in the disease inhibiting agent as will be described later was produced in similar operations as those in the production of FC except that an Aspergillus oryzae extraction enzyme having aminopeptidase N activity was used in the secondary enzymatic reaction.

The FC-2 was analyzed by TLC similarly to the case of FC, and the presence of peptide molecules (POG)₅, (POG)₄, (POG)₃, and (POG)₂ was confirmed.

Further, from the MALDI-TOF/MS analysis, it was confirmed that this FC-2 also contains peptide molecules G(POG)₄, G(POG)₃, and G(POG)₂.

From the ion peak analysis in CID-LIFT of MALDI-TOF/MS, it was revealed that the FC-2 contains 0.01% of (POG)₅, 0.04% of G(POG)₄, 0.1% of (POG)₄, 0.3% of G(POG)₃, 1% of (POG)₃, 2% of G(POG)₂, and 3% of (POG)₂.

[Preparation of Collagen Peptide Containing Peptide Molecule Having a Specific Structure, 6]

Collagen peptide derived from pig skin (PC-CP-2) containing a peptide molecule having a specific structure for use in the performance evaluation tests and in the disease inhibiting agent as will be described later was obtained in similar operations as those in the production of PC-CP except that an Aspergillus niger extraction enzyme having both aminopeptidase N and prolyloligopeptidase activities was used in the secondary enzymatic reaction.

The PC-CP-2 was analyzed by TLC in a similar manner to the case of the PC, and the presence of peptide molecules (POG)₅, (POG)₄, (POG)₃, and (POG)₂ was confirmed.

Further, from the MALDI-TOF/MS analysis, it was confirmed that this PC-CP-2 also contains peptide molecules G(POG)₄, G(POG)₃, and G(POG)₂.

From the ion peak analysis in CID-LIFT of MALDI-TOF/MS, it was revealed that the PC-CP-2 contains 0.02% of (POG)₅, 0.04% of G(POG)₄, 0.2% of (POG)₄, 0.4% of G(POG)₃, 2% of (POG)₃, 4% of G(POG)₂, and 9% of (POG)₂.

[Preparation of Collagen Peptide not Containing Peptide Molecule Having a Specific Structure, 1]

Collagen peptide for comparison (PC-CP-Cont) containing no peptide molecule having a specific structure for use in the performance evaluation tests and in the disease inhibiting agent as will be described later was obtained according to the following method.

In brief, 1 kg of gelatin being a thermal-denatured product of collagen derived from pig skin (Type I collagen) was dissolved in 4 L of 20 mM Tris-HCl buffer (pH 7.5) under warming, and then cooled to 40° C., and then as a primary enzymatic reaction, 1 g of collagenase (produced by Nitta Gelatin Inc., Collagenase N2) was added and the reaction was retained at pH 7.0 to 7.8 at a temperature of 40° C. for 18 hours for an enzymatic decomposition treatment. Then, the solution obtained by the enzymatic hydrolysis treatment was heated at 100° C. for 10 minutes, and then cooled to 60° C., and filtered by using activated charcoal and a filtration aid (diatomaceous earth), and the obtained mother liquor was subjected to a high temperature sterilization treatment at 120° C. for 3 seconds. Then, the sterilized mother liquor was spray-dried to obtain PC-CP-Cont.

The PC-CP-Cont was analyzed by TLC in a similar manner to the case of the PC, and further MALDI-TOF/MS analysis was conducted, but no peptide molecules having a specific structure were observed.

[Performance Evaluation Test]

Details of the performance evaluation tests conducted using each of the foregoing peptide molecules, collagen peptides, and amino acids for comparison (proline, hydroxyproline) will be shown below.

<Evaluation Test 1: Inhibition of Differentiation and Activation of Osteoclast>

Evaluation was made in conformance with an osteoclast differentiation culture method by Kobayashi Y. et al. [J. Bone Miner. Metab. (2004) 22: p. 318-328].

In brief, either of EGDGHLGKPGROGE (SEQ ID NO:1), EKDGHPGKPGROGE (SEQ ID NO:2), (POG)₅, G(POG)₄, (POG)₄, G(POG)₃, (POG)₃, G(POG)₂ or (POG)₂ was added to a mouse primary bone marrow cell culture liquid in a final concentration of 625 μM, and activity of inhibiting tartaric acid-resistant acidic phosphatase (TRAP) being a marker enzyme was examined for each of the peptides after 6 days from culture. Similarly, TRAP inhibiting activity was examined for each of other peptide molecules (PO, Ala-Hyp, Leu-Hyp, Phe-Hyp, Ser-Hyp, POG), and amino acids (Pro, Hyp). As a control, TRAP inhibiting activity when no peptide was added (blank) was also examined.

Further, the inhibition degree of differentiation and activation of osteoclast by each of the peptide molecules, and amino acids was evaluated by the following Pit assay. In brief, the Pi assay in which osteoclast is cultured on an ivory piece was conducted in conformance with Kakudo S, et al J. Bone Miner. Metab. (1996) 14: 129-136. The concrete procedure is as follows.

A suspension containing precursor cells of osteoclasts derived from juvenile mouse intestinal tract bone and bone marrow stromal cells was freeze-preserved at −80° C. in the presence of 10% DMSO to kill matured osteoclasts.

The resultant cells (2.0×10⁵) were seeded in each well of a 96-well plate in which a ivory piece was set, and each peptide to be tested was added to the culture liquid, and cultured at 37° C., 5% CO₂ for about 1 week. Then, after removing the cells from the ivory piece with a silicone rubber policeman, the ivory piece was stained with an acid hematoxylin solution for several minutes. At this time, the number of TRAP staining positive multinucleated giant cells (osteoclast) was counted by TRAP staining, and a relative number with respect to the number of cells in control (blank) was calculated. Then, the Pit number (number of resorption cavity) by osteoclast was counted under a microscope, and the degree of inhibiting osteoclast by each peptide to be tested was indicated by a relative ratio to blank (control).

The result is shown in Table 1.

TABLE 1

| | Relative number of TRAP- | Relative area of TRAP- | Relative number of TRAP- | |
|---|---|---|---|---|
| | positive multinucleated | positive multinucleated | positive multinucleated | |
| | giant cells (osteoclasts) | giant cells (osteoclasts) | giant cells (osteoclasts) | |
| | by cultivation on plastic | by cultivation on plastic | by cultivation on ivory | Relative number |
| | dish (%) | dish (%) | piece (%) | of Pit (%) |
| Control (blank) | 100 | 100 | 100 | 100 |
| EGDGHLGKPGROGE | 3 ± 2** | 2 ± 1** | 4 ± 3** | 2 ± 1** |
| (SEQ ID NO: 1) | | | | |
| EKDGHPGKPGROGE | 11 ± 6** | 10 ± 5** | 13 ± 4** | 10 ± 4** |
| (SEQ ID NO: 2) | | | | |
| (POG)₅ | 5 ± 1** | 3 ± 1** | 5 ± 3** | 2 ± 1** |
| G(POG)₄ | 13 ± 2** | 12 ± 3** | 17 ± 4** | 11 ± 3** |
| (POG)₄ | 2 ± 1** | 1 ± 1** | 3 ± 2** | 2 ± 1** |
| G(POG)₃ | 10 ± 2** | 10 ± 2** | 19 ± 4** | 16 ± 5** |
| (POG)₃ | 4 ± 2** | 2 ± 1** | 4 ± 3** | 3 ± 1** |
| G(POG)₂ | 11 ± 3** | 10 ± 3** | 16 ± 5** | 18 ± 3** |
| (POG)₂ | 3 ± 1** | 2 ± 1** | 3 ± 2** | 3 ± 1** |
| OG | 9 ± 2** | 7 ± 1** | 9 ± 5** | 2 ± 2** |
| PO | 130 ± 9* | 120 ± 12* | 17 ± 6** | 9 ± 2** |
| Ala-Hyp | 102 ± 4 | 110 ± 31 | 89 ± 13 | 101 ± 12 |
| Leu-Hyp | 88 ± 22 | 83 ± 27 | 101 ± 12 | 91 ± 11 |
| Phe-Hyp | 119 ± 16 | 118 ± 21 | 98 ± 11 | 109 ± 15 |
| Ser-Hyp | 96 ± 5 | 91 ± 10 | 105 ± 4 | 98 ± 12 |
| POG | 109 ± 15 | 113 ± 11 | 91 ± 11 | 97 ± 13 |
| Pro | 119 ± 44 | 125 ± 69 | 119 ± 20 | 121 ± 23 |
| Hyp | 126 ± 4* | 117 ± 13* | 141 ± 9* | 131 ± 11* |

(Test number: n = 6)
Note)
**Statistically significant difference in comparison with control (p < 0.01)
*Statically significant difference in comparison with control (p < 0.05)

<Evaluation Test 2: Promotion of Differentiation and Activation of Osteoblast>

Each of dexamethasone (final concentration 1 nmol/L), β-glycerophosphate (final concentration 5 mmol/L), and ascorbic acid (final concentration 100 μg/mL) was added to an osteoblast strain MC3T3-E1 culture solution, and then either of EGDGHLGKPGROGE (SEQ ID NO:1), EKDGHPGKPGROGE (SEQ ID NO:2), (POG)₅, G(POG)₄, (POG)₄, G(POG)₃, (POG)₃, G(POG)₂ or (POG)₂ was added to the culture liquid so as to be a final concentration of 2.5 mmol/L, and after 10 days from culture, activity of promoting alkaline phosphatase (ALP) being a marker enzyme for differentiation and activation of osteoblast was examined for each of the peptides. Similarly, ALP promoting activity was examined for each of other peptide molecules (PO, Ala-Hyp, Leu-Hyp, Phe-Hyp, Ser-Hyp, POG)), and amino acids (Pro, Hyp). Further, as a control, ALP promoting activity when no peptide was added (blank) was also examined. The result is shown in Table 2.

TABLE 2

| | Relative value of ALP (%) |
|---|---|
| Control (blank) | 100 |
| EGDGHLGKPGROGE | 174 ± 9** |
| (SEQ ID NO: 1) | |
| EKDGHPGKPGROGE | 157 ± 12* |
| (SEQ ID NO: 2) | |
| (POG)₅ | 143 ± 21** |
| G(POG)₄ | 120 ± 9** |
| (POG)₄ | 157 ± 12** |
| G(POG)₃ | 121 ± 8* |
| (POG)₃ | 165 ± 10** |
| G(POG)₂ | 124 ± 7* |
| (POG)₂ | 169 ± 9** |
| OG | 140 ± 24** |
| PO | 115 ± 25 |
| Ala-Hyp | 112 ± 31 |
| Leu-Hyp | 92 ± 12 |
| Pbe-Hyp | 109 ± 11 |
| Ser-Hyp | 91 ± 21 |
| POG | 103 ± 22 |
| Pro | 97 ± 15 |
| Hyp | 103 ± 25 |

(Test number: n = 6)
Note)
**Statistically significant difference in comparison with control (p < 0.01)
*Statically significant difference in comparison with control (p < 0.05)

<Evaluation Test 3. Inhibition of Degeneration of Chondrocyte>

Either of EGDGHLGKPGROGE (SEQ ID NO:1), EKDGHPGKPGROGE (SEQ ID NO:2), (POG)₅, G(POG)₄, (POG)₄, G(POG)₃, (POG)₃, G(POG)₂, or (POG)₂ was added to a chondrocyte precursor strain ATDC5 culture liquid in a final concentration of 2.5 mmol/L, and after 5 days from the culture, activity of inhibiting alkaline phosphatase (ALP) being a marker enzyme for enlarged cartilage and calcification was examined for each of the peptides. Similarly, ALP activity was examined for each of other peptide molecules (PO, Ala-Hyp, Leu-Hyp, Phe-Hyp, Ser-Hyp, POG), and amino acids (Pro, Hyp). Further, as a control, ALP activity when no peptide was added (blank) was also examined. The result is shown in Table 3.

TABLE 3

| | Relative value of ALP (%) |
|---|---|
| Control (blank) | 100 |
| EGDGHLGKPGROGE | 35 ± 3** |
| (SEQ ID NO: 1) | |
| EKDGHPGKPGROGE | 38 ± 5** |
| (SEQ ID NO: 2) | |
| (POG)₅ | 78 ± 6* |
| G(POG)₄ | 80 ± 7* |
| (POG)₄ | 73 ± 9* |
| G(POG)₃ | 81 ± 6* |
| (POG)₃ | 76 ± 5* |
| G(POG)₂ | 78 ± 9* |
| (POG)₂ | 77 ± 8* |
| OG | 76 ± 21* |
| PO | 12 ± 2** |
| Ala-Hyp | 17 ± 6** |
| Leu-Hyp | 93 ± 12 |
| Pbe-Hyp | 109 ± 11 |
| Ser-Hyp | 91 ± 21 |
| POG | 84 ± 14 |
| Pro | 98 ± 10 |
| Hyp | 101 ± 1 |

(Test number: n = 6)
Note)
**Statistically significant difference in comparison with control (p < 0.01)
*Statically significant difference in comparison with control (p < 0.05)

<Evaluation Test 4: Recovery of Tropocollagen Amount in Dermis of Skin>

After preliminarily feeding Male Wistar rat (140 g) with a commercially available solid food (TypeMF, produced by Oriental Yeast Co., Ltd.) for three days, the feed was changed to casein food, and skin wound was allowed to develop after three days.

The skin wound was allowed to develop by conducting a depilatory treatment on the abdominal area of the rat for three days, and concretely, the rat was anesthetized by intraperitoneal administration of Nembutal (4 mg/0.08 mL/100 g BW), and then the abdominal area (about 3×5 cm) was sheared by an electric shaver. Further, a commercially available depilatory (Epilat depilatory cream, produced by Kanebo) was applied, and left for 5 minutes, and shaved carefully with a razor. This treatment was conducted once a day continuously for three days since three days before start of collecting a skin sample.

The test groups were separated into the following groups: casein food group, EGDGHLGKPGROGE (SEQ ID NO:1) group, EKDGHPGKPGROGE (SEQ ID NO:2) group, (POG)₅ group, G(POG)₄ group, (POG)₄ group, G(POG)₃ group, (POG)₃ group, G(POG)₂ group, (POG)₂ group, PC group, FC group, PC-CP group, PC-2 group, FC-2 group and PC-CP-2 group; and transition of the skin collagen amount in the skin wound recovery process (percentage per total collagen amount) was measured for each group at the day of the depilatory treatment (at day 0 after the depilatory treatment), one day after the depilatory treatment, two days after the depilatory treatment and four days after the depilatory treatment. Feed compositions of respective groups are shown in Table 4.

TABLE 4

| Ingredient | Peptide molecule having a specific structure | | | | | | | | |
|---|---|---|---|---|---|---|---|---|---|
| Casein | 145 | 145 | 145 | 145 | 145 | 145 | 145 | 145 | 145 |
| (POG)₅ | 5 | — | — | — | — | — | — | — | — |
| G(POG)₄ | — | 5 | — | — | — | — | — | — | — |
| (POG)₄ | — | — | 5 | — | — | — | — | — | — |
| G(POG)₃ | — | — | — | 5 | — | — | — | — | — |
| (POG)₃ | — | — | — | — | 5 | — | — | — | — |
| G(POG)₂ | — | — | — | — | — | 5 | — | — | — |
| (POG)₂ | — | — | — | — | — | — | 5 | — | — |
| EGDGHLGKPGROGE | — | — | — | — | — | — | — | 5 | — |
| (SEQ ID NO: 1) | | | | | | | | | |
| EKDGHPGKPGROGE | — | — | — | — | — | — | — | — | 5 |
| (SEQ ID NO: 2) | | | | | | | | | |
| α-cornstarch | 735 | 735 | 735 | 735 | 735 | 735 | 735 | 735 | 735 |
| Corn oil | 50 | 50 | 50 | 50 | 50 | 50 | 50 | 50 | 50 |
| Cellulose | 20 | 20 | 20 | 20 | 20 | 20 | 20 | 20 | 20 |
| Mineral mixture | 35 | 35 | 35 | 35 | 35 | 35 | 35 | 35 | 35 |
| Vitamin mixture | 10 | 10 | 10 | 10 | 10 | 10 | 10 | 10 | 10 |
| Total | 1000 | 1000 | 1000 | 1000 | 1000 | 1000 | 1000 | 1000 | 1000 |

| | Control | | | | | | | |
|---|---|---|---|---|---|---|---|---|
| | (Casein | | | | | | | |
| Ingredient | food) | PC | FC | PC-CP | PC-2 | FC-2 | PC-CP-2 | OG |
| Casein | 150 | 100 | 100 | 100 | 100 | 100 | 100 | 145 |
| PC | — | 50 | — | — | — | — | — | — |
| FC | — | — | 50 | — | — | — | — | — |
| PC-CP | — | — | — | 50 | — | — | — | — |
| PC-2 | — | — | — | — | 50 | — | — | — |
| FC-2 | — | — | — | — | — | 50 | — | — |
| PC-CP-2 | — | — | — | — | — | — | 50 | — |
| OG | — | — | — | — | — | — | — | 5 |
| α-cornstarch | 735 | 735 | 735 | 735 | 735 | 735 | 735 | 735 |
| Corn oil | 50 | 50 | 50 | 50 | 50 | 50 | 50 | 50 |
| Cellulose | 20 | 20 | 20 | 20 | 20 | 20 | 20 | 20 |
| Mineral mixture | 35 | 35 | 35 | 35 | 35 | 35 | 35 | 35 |
| Vitamin mixture | 10 | 10 | 10 | 10 | 10 | 10 | 10 | 10 |
| Total | 1000 | 1000 | 1000 | 1000 | 1000 | 1000 | 1000 | 1000 |

The rats were fed with the aforementioned feed compositions, and allowed to take a feed and water ad libitum throughout the feeding period.

Further, in EGDGHLGKPGROGE (SEQ ID NO:1) group, EKDGHPGKPGROGE (SEQ ID NO:2) group, (POG)₅ group, G(POG)₄ group, (POG)₄ group, G(POG)₃ group, (POG)₃ group, G(POG)₂ group, (POG)₂ group, PC group, FC group, PC-CP group, PC-2 group, FC-2 group, and PC-CP-2 group, 10 g of the same as each of the specific peptide molecules PC, FC, PC-CP, PC-2, FC-2, and PC-CP-2 blended in the feed was accurately weighed, and dissolved in 20 mL of distilled water while it was kept warm, and then intragastrically administered to a rat of each test group once a day at noon by using a sonde.

A measurement result of transition of the skin collagen amount in the skin wound recovery process (percentage per total collagen amount) of each group is shown in Table 5.

TABLE 5

| | Transition of skin collagen amount in skin wound recovery process | | | | |
|---|---|---|---|---|---|
| | (Raio to total collagen amount)(%) | | | | |
| | | 0 day after | 1 day after | 2 days after | 4 days after |
| | | depilatory | depilatory | depilatory | depilatory |
| | No treatment | treatment | treatment | treatment | treatment |
| Control (Casein food) | 8.2 ± 0.6^{a} | 2.9 ± 0.3^{b} | 2.5 ± 0.2^{b} | 2.6 ± 0.3^{b} | 3.1 ± 0.4^{b} |
| EGDGHLGKPGROGE | 8.2 ± 0.6^{a} | 2.4 ± 0.3^{b} | 2.7 ± 0.4^{b} | 2.9 ± 0.2^{bc} | 5.2 ± 0.4^{d} |
| (SEQ ID NO: 1) | | | | | |
| EKDGHPGKPGROGE | 8.2 ± 0.6^{a} | 2.5 ± 0.1^{b} | 2.7 ± 0.3^{b} | 3.1 ± 0.3^{c} | 5.2 ± 0.2^{d} |
| (SEQ ID NO: 2) | | | | | |
| (POG)₅ | 8.2 ± 0.6^{a} | 2.1 ± 0.4^{b} | 2.4 ± 0.3^{b} | 3.0 ± 0.2^{c} | 5.0 ± 0.4^{d} |
| G(POG)₄ | 8.2 ± 0.6^{a} | 2.2 ± 0.3^{b} | 2.4 ± 0.3^{b} | 2.9 ± 0.4^{c} | 4.3 ± 0.3^{d} |
| (POG)₄ | 8.2 ± 0.6^{a} | 2.4 ± 0.3^{b} | 2.9 ± 0.1^{c} | 3.3 ± 0.3^{c} | 5.1 ± 0.2^{d} |
| G(POG)₃ | 8.2 ± 0.6^{a} | 2.2 ± 0.3^{b} | 2.5 ± 0.4^{b} | 2.9 ± 0.2^{c} | 4.4 ± 0.3^{d} |
| (POG)₃ | 8.2 ± 0.6^{a} | 2.3 ± 0.2^{b} | 2.9 ± 0.3^{c} | 3.3 ± 0.2^{c} | 5.2 ± 0.2^{d} |
| G(POG)₂ | 8.2 ± 0.6^{a} | 2.2 ± 0.2^{b} | 2.5 ± 0.1^{c} | 2.8 ± 0.2^{c} | 4.5 ± 0.2^{d} |
| (POG)₂ | 8.2 ± 0.6^{a} | 2.3 ± 0.3^{b} | 3.0 ± 0.2^{c} | 3.5 ± 0.3^{c} | 5.3 ± 0.3^{d} |
| PC | 8.2 ± 0.6^{a} | 2.1 ± 0.3^{b} | 2.3 ± 0.1^{c} | 3.1 ± 0.3^{c} | 4.6 ± 0.2^{d} |
| FC | 8.2 ± 0.6^{a} | 2.6 ± 0.3^{b} | 2.5 ± 0.3^{b} | 3.5 ± 0.2^{c} | 4.5 ± 0.4^{d} |
| PC-CP | 8.2 ± 0.6^{a} | 2.5 ± 0.1^{b} | 3.1 ± 0.4^{c} | 3.8 ± 0.1^{c} | 5.1 ± 0.2^{d} |
| PC-2 | 8.2 ± 0.6^{a} | 2.1 ± 0.2^{b} | 2.4 ± 0.3^{c} | 3.2 ± 0.2^{c} | 4.7 ± 0.2^{d} |
| FC-2 | 8.2 ± 0.6^{a} | 2.6 ± 0.2^{b} | 2.5 ± 0.2^{c} | 3.6 ± 0.3^{c} | 4.6 ± 0.5^{d} |
| PC-CP-2 | 8.2 ± 0.6^{a} | 2.4 ± 0.2^{b} | 3.2 ± 0.3^{c} | 3.9 ± 0.2^{c} | 5.3 ± 0.3^{d} |
| OG | 8.2 ± 0.6^{a} | 2.4 ± 0.2^{b} | 3.1 ± 0.3^{c} | 4.0 ± 0.2^{c} | 5.2 ± 0.3^{d} |

(Subject animal number: n = 4)
Note)
Statistically significant difference between different alphabetical characters (p < 0.05)
(Annotation): Ratio of skin tropocollagen (%) = X ÷ [X + Y + Z] × 100
X: Amount of collagen soluble to aqueous 0.45M NaCl solution: Tropocollagen amount
Y: Amount of collagen soluble to aqueous 0.5M acetic acid solution: Acid soluble collagen amount
Z: Amount of collagen insoluble to aqueous 0.5M acetic acid solution: (acid insoluble collagen = cross-linked collagen) amount

Here, quantification of skin soluble collagen was conducted in the following manner.

Treated skin and untreated skin were trimmed while fat under each skin was removed as much as possible. Each skin was cut finely with a dissecting scissor deliberately, and approximately 0.2 to 0.3 g was finely weighed, and collected in a 14 mL-volume centrifugal tube. Then, 4 mL of a cold 0.45 M sodium chloride solution was added and homogenized by a Polytron homogenizer (speed No4) for 20 seconds under ice cooling. Further, 2 mL of a cold 0.45 M sodium chloride solution was added, and extraction was conducted for 24 hours in a refrigerator using a rotary stirrer (manufactured by TAITEC). The extract was centrifuged at 20,000 g for 20 minutes by a refrigerated centrifuge, and the supernatant liquid was collected and named a neutral salt-soluble collagen fraction. To the residue of the centrifugation was added 6 mL of cold 0.5 M acetic acid, and extraction was conducted similarly for 24 hours. The liquid extracted with 0.5 M acetic acid was centrifuged at 20,000 g for 20 minutes by a refrigerated centrifuge, and the supernatant liquid was collected and named an acid soluble collagen fraction. The residue of the centrifugation was named an insoluble collagen fraction.

To 5 mL of each of the neutral salt-soluble collagen fraction and the acid soluble collagen fraction were respectively added an equivalent volume, 5 mL of concentrated hydrochloric acid, and to the insoluble collagen fraction was added 1 mL of concentrated hydrochloric acid. Each collagen fraction was dissolved at 60° C. for five minutes under warming, and transferred to a glass test tube for hydrolysis while washed three times with 2 mL of 6 N hydrochloric acid, and hydrolyzed at 110° C. for 24 hours.

Then, the amount of hydroxyproline contained in the hydrolysis liquid of each collagen fraction was colorimetrically quantified, to achieve quantification of each collagen fraction, and a relative ratio of the neutral salt-soluble collagen fraction to the sum of these collagen fractions was calculated.

The colorimetric quantification of the amount of hydroxyproline was conducted by a Firschein and Shill method, and was concretely conducted in the following manner.

Two mL of 2-propanol was added to 2 mL of a sample solution and stirred thoroughly. Then, 0.5 mL of a chloramine T liquid being an oxidizing agent was added, and left still for accurately 4 minutes, and then cooled on ice. Then, 5 mL of a p-dimethylaminobenzaldehyde solution was added and stirred thoroughly, and then heated in a boiling water bath for accurately 2 minutes. Then, the reaction was immediately cooled on ice, and left still for 1 hour, and then colorimetrically quantified at a wavelength of 575 nm.

As the chloramine T liquid, a solution prepared by dissolving chloramine T (5 g) in 50 mL of distilled water was stored in a refrigerator, and a liquid prepared by diluting the solution with acetic acid buffer (pH 6.0) at a ratio of 1:4 directly before use was used. Further, the p-dimethylaminobenzaldehyde solution (Erich solution) was prepared by dissolving 20 g of p-dimethylaminobenzaldehyde powder in 22 mL of concentrated hydrochloric acid under heating in boiling water, and immediately cooling the same in ice water, and adding 122 mL of 2-propanol and dissolving it under stirring.

<Evaluation Test 5: Intestinal Tract Absorptivity>

Male Wistar rats (170 g) were fasted overnight before subjected to the experiment. As a test sample, 215 nmol/10 mL of each of EGDGHLGKPGROGE (SEQ ID NO:1), EKDGHPGKPGROGE (SEQ ID NO:2), (POG)₅, G(POG)₄, (POG)₄, G(POG)₃, (POG)₃, G(POG)₂, (POG)₂, OG, PO, Ala-Hyp, and Ser-Hyp was used, and intragastrically administered.

As a test method, heart and portal vein of each rat were attached with a cannula to make one-directional perfusion. As a perfusate, a Krebs-Ringer bicarbonic acid liquid (KRB liquid, pH 7.4) composed of 9.0 g of NaCl, 8 mL of 5.75% KCl, 2 mL of 10.55% KH₂PO₄, 2 mL of 19% MgSO₄, 2.73 g of NaHCO₃, 3.43 g of glucose, and 1255 mL of water, and to which were added 10 g of bovine serum albumin, 0.5 mL of dexamethasone (0.123 mg/mL) and 0.5 mL of noradrenaline (0.024 mg/mL) per 500 mL of the KRB liquid was used.

To a perfusion sample solution (5.0 mL) collected from the portal vein was added 0.5 mL of 30% sulfosalicylic acid and stirred vigorously, and left overnight in a refrigerator. This sample was centrifuged at 3000 rpm for 10 minutes, to remove protein. For the supernatant of centrifugation, an amount of hydroxyproline in 0.5 mL was colorimetrically quantified, and an amount of free-type Hyp was obtained.

Further, 3.0 mL of the supernatant of centrifugation was weighed into a screw-top test tube, and thereto an equivalent amount of concentrated hydrochloric acid was added, and hydrolyzed at 110° C. for 24 hours. After concentrating and drying the resultant in an evaporator, and removing the hydrochloric acid, the solid was dissolved in 5 mL of distilled water, and several drops of a saturated lithium hydroxide solution was added thereto to adjust pH at 5 to 7, and the volume was fixed at 10 mL. For 2 mL of this solution, an amount of hydroxyproline was colorimetrically quantified to obtain a total Hyp amount. The value obtained by subtracting the amount of free-type Hyp before hydrolysis from the total Hyp amount after hydrolysis is an amount of peptide-form Hyp. From this amount of peptide-form Hyp, a quantitative value of absorption of each peptide molecule into rat portal vein perfusate in the test sample was first determined.

In the above description, the colorimetric quantification of the amount of hydroxyproline was conducted by the Firschein and Shill method described concretely in Evaluation test 4.

Further, the peptide molecule recovered into rat portal vein perfusate, namely each of EGDGHLGKPGROGE (SEQ ID NO:1), EKDGHPGKPGROGE (SEQ ID NO:2), (POG)₅, G(POG)₄, (POG)₄, G(POG)₃, (POG)₃, G(POG)₂, and (POG)₂ was identified and quantified by the MALDI-TOF/MS analysis. Also, identification and quantification of OG, PO, Ala-Hyp, and Ser-Hyp were conducted by HPLC analysis and mass spectrometry (LC/MS/MS) as will be described later.

(HPLC Analysis)

Analysis of the peptide molecules in the perfusate was conducted by reverse-phase HPLC analysis. As a HPLC device, an LCSS-905 system manufactured by JASCO Corporation, consisting of a liquid feeding pump, a degasser, an automatic sampler, a column open, a UV spectrophotometer, a printer, and a system controller was used. As a reverse-phase column, Nova Pak C18 (3.9×150 mm) was used.

A linear gradient mobile phase of a 0.1% TFA-containing acetonitrile-water system was used, and the injection amount of the sample was 70 μL and the flow rate was 1 mL/min.

(LC/MS/MS Analysis)

As a HPLC device, U980HPLC (manufactured by JASCO Corporation) attached with an ODS(C18) column (Mightysil RP-18, 2×250 mm, manufactured by Kanto Chemical Co Ltd) was used. As a mobile phase solvent, a 0.2% formic acid-containing acetonitrile-water system was used, and the concentration of acetonitrile was increased from 0% to 40% over 40 minutes by a linear gradient, and washed with 100% acetonitrile for 10 minutes. The sample injection amount was 10 μL, and the column temperature was 40° C.

MS analysis was conducted by a MS/MS system using a Quattro LC mass spectrophotometer (Micromass, Manchester, UK) according to a four-channel Multiple Reaction Monitoring method. To be more specific, the elute from HPLC was monitored by m/z being [M+H]⁺ and by m/s of its fragment ion species. The monitoring was conducted by using [M+H]⁺ m/z: 229.1>132.1 for PO, [M+H]⁺ m/z: 219.1>132.1 for Ser-Hyp, [M+H]⁺ m/z: 203.1>132.1 for Ala-Hyp, and [M+H]⁺ m/z: 189.1>86.1 for OG.

The perfusate was treated with sulfosalicylic acid in a final concentration of 3%, to remove protein. The supernatant liquid was lyophilized and 10 mg of a dry powder was dissolved in distilled water, and subjected to a positive ion exchange resin column to obtain an ammonia elution fraction. After removing the solvent, the fraction was dissolved in distilled water and subjected to LC/MS/MS analysis.

The result is shown in Table 6.

TABLE 6

| Administered peptide | Amount of each peptide molecule identified |
|---|---|
| molecule | after absorption (nmol/mL) |
| EGDGHLGKPGROGE | 0.1 |
| (SEQ ID NO: 1) | |
| EKDGHPGKPGROGE | 0.1 |
| (SEQ ID NO: 2) | |
| (POG)₅ | 0.02 |
| G(POG)₄ | 0.05 |
| (POG)₄ | 0.8 |
| G(POG)₃ | 0.08 |
| (POG)₃ | 0.9 |
| G(POG)₂ | 0.1 |
| (POG)₂ | 1.1 |
| OG | 9.8 |
| PO | 21.3 |
| Ala-Hyp | 1.2 |
| Ser-Hyp | 0.7 |

<Evaluation Test 6>

Ten-week old C57BL/6J mice were allowed to orally take respective feeds having the compositions shown in the following Table 7.

TABLE 7

| | | C | Peptide molecule having a specific structure-added | | | |
|---|---|---|---|---|---|---|
| | N group | group | group | | | |
| Casein | 200 | 200 | 200 | 200 | 200 | 200 |
| Lard | 58.3 | 58.3 | 58.3 | 58.3 | 58.3 | 58.3 |
| Corn oil | 11.7 | 11.7 | 11.7 | 11.7 | 11.7 | 11.7 |
| Mineral mixture | 35 | 35 | 35 | 35 | 35 | 35 |
| Vitamin mixture | 10 | 10 | 10 | 10 | 10 | 10 |
| Sucrose | 100 | 100 | 100 | 100 | 100 | 100 |
| Corn starch | 529.5 | 470.45 | 517.45 | 517.45 | 517.45 | 517.45 |
| Cellulose | 50 | 50 | 50 | 50 | 50 | 50 |
| L-cystine | 3 | 3 | 3 | 3 | 3 | 3 |
| Potassium phosphate | — | 59.05 | 59.05 | 59.05 | 59.05 | 59.05 |
| (POG)₅ | — | — | 3 | — | — | — |
| G(POG)₄ | — | — | — | 3 | — | — |
| (POG)₄ | — | — | — | — | 3 | — |
| G(POG)₃ | — | — | — | — | — | 3 |
| | | | | | | OG added |
| | Peptide molecule having a specific structure-added group | | | | | group |
| Casein | 200 | 200 | 200 | 200 | 200 | 200 |
| Lard | 58.3 | 58.3 | 58.3 | 58.3 | 58.3 | 58.3 |
| Corn oil | 11.7 | 11.7 | 11.7 | 11.7 | 11.7 | 11.7 |
| Mineral mixture | 35 | 35 | 35 | 35 | 35 | 35 |
| Vitamin mixture | 10 | 10 | 10 | 10 | 10 | 10 |
| Sucrose | 100 | 100 | 100 | 100 | 100 | 100 |
| Corn starch | 517.45 | 517.45 | 517.45 | 517.45 | 517.45 | 517.45 |
| Cellulose | 50 | 50 | 50 | 50 | 50 | 50 |
| L-cystine | 3 | 3 | 3 | 3 | 3 | 3 |
| Potassium phosphate | 59.05 | 59.05 | 59.05 | 59.05 | 59.05 | 59.05 |
| (POG)₃ | 3 | — | — | — | — | — |
| G(POG)₂ | — | 3 | — | — | — | — |
| (POG)₂ | — | — | 3 | — | — | — |
| EGDGHLGKPGROGE | — | — | — | 3 | — | — |
| (SEQ ID NO: 1) | | | | | | |
| EKDGHPGKPGROGE | — | — | — | — | 3 | — |
| (SEQ ID NO: 2) | | | | | | |
| OG | — | — | — | — | — | 3 |

Mice were sacrificed after three weeks, and from a μCT (desktop micro CT scanner SKYSCAN1172, manufactured by SKYSCAN) image of a femur-tibia joint of each group, width of the joint space was measured, and from a non-decalcified hematoxylin staining section, a matrix structure was evaluated and a cell condition was evaluated.

The result is shown in Table 8.

TABLE 8

| | | | EGDGHLGKPGROGE | EKDGHPGKPGROGE | | |
|---|---|---|---|---|---|---|
| | N group | C group | (SEQ ID NO: 1) | (SEQ ID NO: 2) | (POG)₅ | G(POG)₄ |
| Relative thickness of | 1.0 ± 0.2 | 0.5 ± 0.1(*) | 0.9 ± 0.2 | 0.9 ± 0.1 | 0.9 ± 0.2 | 0.7 ± 0.1 |
| articular cartilage | | | | | | |
| Pathological score | 0.2 ± 0.04 | 5.0 ± 1.5(*) | 0.3 ± 0.1 | 0.2 ± 0.07 | 0.3 ± 0.1 | 0.5 ± 0.3 |
| (articular cartilage part) | | | | | | |
| Characteristic pathological | — | Significant | Trabecula similar to that in | Trabecula similar to that in | Trabecula similar | Same as on |
| finding in joint cancellous | | decrease in | N group. Equivalent | N group. Equivalent | to that in N | the left. |
| bone part in comparison | | trabecula. | numbers of osteoblasts and | number of osteoblasts to | group. | |
| with N group | | Significant | bone cells to those in N | that in N group present. | Equivalent | |
| | | decrease in | group present. | | numbers of | |
| | | osteoblast and | | | osteoblasts and | |
| | | bone cell, and | | | bone cells to | |
| | | increase | | | those in N group | |
| | | number of | | | present. | |
| | | osteoclasts. | | | | |
| | (POG)₄ | G(POG)₃ | (POG)₃ | G(POG)₂ | (POG)₂ | OG |
| Relative thickness of | 0.9 ± 0.1 | 0.7 ± 0.2 | 0.9 ± 0.1 | 0.8 ± 0.2 | 1.0 ± 0.2 | 1.0 ± 0.2 |
| articular cartilage | | | | | | |
| Pathological score | 0.3 ± 0.05 | 0.5 ± 0.3 | 0.2 ± 0.05 | 0.4 ± 0.2 | 0.2 ± 0.1 | 0.3 ± 0.05 |
| (Articular cartilage part) | | | | | | |
| Characteristic pathological | Trabecula similar | Same as on the | Same as on the left. | Same as on the left. | Same as on the | Same as on |
| finding in joint cancellous | to that in N | left. | | | left. | the left. |
| bone part in comparison | group. | | | | | |
| with N group | Equivalent | | | | | |
| | numbers of | | | | | |
| | osteoblasts and | | | | | |
| | bone cells to | | | | | |
| | those in N group | | | | | |
| | present. | | | | | |

Subject animal number: n = 4
Note
*Statistically significant difference in comparison with N group (p < 0.05

<Evaluation Test 7>

After solubilizing each of (POG)₅, G(POG)₄, (POG)₄, G(POG)₃, (POG)₃, G(POG)₂, and (POG)₂ in a final concentration of 5 mmol/L in saline, the solution was sterilized by filtration. Each of these solutions (0.5 mL) was injected to the left femur-tibia joint space for C group, 10-week old C57BL/6J mice fed with the feed having the composition shown in Table 7 for three weeks. The mice were sacrificed after one week, and non-decalcified Mayer's hematoxylin staining sections of the left and right femur-tibia joint spaces were prepared, and evaluated pathologically. In a similar manner, for the case where the mice were sacrificed after three weeks from injection, the non-decalcified Mayer's hematoxylin staining sections of the left and right femur-tibia joint spaces were prepared, and evaluated pathologically in comparison with the pathological sections of N group in the foregoing Evaluation test 6.

The result is shown in Table 9.

TABLE 9

| | | (POG)₅ group | | G(POG)₄ group | |
|---|---|---|---|---|---|
| | N group | After 1 week | After 3 weeks | After 1 week | After 3 weeks |
| Relative thickness of | 1.0 ± 0.2 | 0.8 ± 0.3 | 1.0 ± 0.1 | 0.7 ± 0.3 | 0.9 ± 0.1 |
| articular cartilage | | | | | |
| Pathological score | 0.2 ± 0.04 | 0.5 ± 0.05 | 0.2 ± 0.03 | 0.6 ± 0.07 | 0.3 ± 0.1 |
| (articular cartilage | | | | | |
| part) | | | | | |
| Characteristic | — | a) | b) | a) | b) |
| pathological finding in | | | | | |
| joint cancellous bone | | | | | |
| part in comparison | | | | | |
| with N group | | | | | |

| | (POG)₄ group | | G(POG)₃ group | | (POG)₃ group | |
|---|---|---|---|---|---|---|
| | | After 3 | | After 3 | After 1 | After 3 |
| | After 1 week | weeks | After 1 week | weeks | week | weeks |
| Relative thickness of | 0.8 ± 0.2 | 1.0 ± 0.1 | 0.7 ± 0.8 | 0.9 ± 0.1 | 0.8 ± 0.2 | 1.0 ± 0.1 |
| articular cartilage | | | | | | |
| Pathological score | 0.5 ± 0.04 | 0.2 ± 0.03 | 0.6 ± 0.06 | 0.3 ± 0.02 | 0.5 ± 0.04 | 0.2 ± 0.03 |
| (articular cartilage | | | | | | |
| part) | | | | | | |
| Characteristic | a) | b) | a) | b) | a) | b) |
| pathological finding in | | | | | | |
| joint cancellous bone | | | | | | |
| part in comparison | | | | | | |
| with N group | | | | | | |

| | G(POG)₂ group | | (POG)₂ group | | OG group | |
|---|---|---|---|---|---|---|
| | | After 3 | | After 3 | After 1 | After 3 |
| | After 1 week | weeks | After 1 week | weeks | week | weeks |
| Relative thickness of | 0.7 ± 0.2 | 0.9 ± 0.1 | 0.8 ± 0.2 | 1.0 ± 0.1 | 0.8 ± 0.2 | 1.0 ± 0.1 |
| articular cartilage | | | | | | |
| Pathological score | 0.6 ± 0.04 | 0.3 ± 0.04 | 0.4 ± 0.07 | 0.2 ± 0.02 | 0.4 ± 0.04 | 0.2 ± 0.03 |
| (articular cartilage | | | | | | |
| part) | | | | | | |
| Characteristic | a) | b) | a) | b) | a) | b) |
| pathological finding in | | | | | | |
| joint cancellous bone | | | | | | |
| part in comparison | | | | | | |
| with N group | | | | | | |

| | EGDGHLGKPGROGE group | | EKDGHPGKPGROGE group | |
|---|---|---|---|---|
| | (SEQ ID NO: 1) | | (SEQ ID NO: 2) | |
| | After 1 week | After 3 weeks | After 1 week | After 3 weeks |
| Relative thickness of | 0.8 ± 0.3 | 1.0 ± 0.3 | 0.8 ± 0.3 | 1.0 ± 0.2 |
| articular cartilage | | | | |
| Pathological score | 0.5 ± 0.07 | 0.2 ± 0.05 | 0.5 ± 0.05 | 0.2 ± 0.04 |
| (articular cartilage | | | | |
| part) | | | | |
| Characteristic | a) | b) | a) | b) |
| pathological finding in | | | | |
| joint cancellous bone | | | | |
| part in comparison | | | | |
| with N group | | | | |

Subject animal number: n = 4
a) Increase in trabecula. Presence of abundant osteoblasts.
b) Similar trabecula to N group. Presence of equivalent numbers of osteoblasts and bone cells to N group.

<Discussion about Result of Performance Evaluation Test>

As can be seen from the above result, comparison with a control (blank) reveals that the peptide molecule having a specific structure inhibits differentiation and activation of osteoclast (Table 1), promotes differentiation and activation of osteoblast (Table 2), inhibits degeneration of chondrocyte to modulate differentiation thereof (Table 3), and recovers the tropocollagen amount in the skin dermis. Its effects are superior to those by peptide molecules other than OG, and by amino acids.

It is also revealed that the peptide molecule having a specific structure is intestinally absorbed sufficiently immediately and stably (without decomposition into amino acids) although not so much as dipeptides (Table 6).

Then, the results shown in Tables 8 and 9 reveal that the peptide molecule having a specific structure inhibits degeneration of articular cartilage, or promotes regeneration of articular cartilage.

[Disease Inhibiting Agent]

Using the peptide molecule having a specific structure, the disease inhibiting agent according to the present invention was obtained. The blending examples thereof are shown below.

### Examples 1 to 7

The ingredients in the blending shown in Table 10 were mixed, and crystalline cellulose as an excipient was used in a proportion of 10 parts with respect to the entirety of the blending described in Table 10, and formed into a tablet according to a routine method, to obtain the disease inhibiting agents according to Example 1 to 7 that can be used for oral administration.

TABLE 10

| | Example 1 | Example 2 | Example 3 | Example 4 | Example 5 | Example 6 | Example 7 |
|---|---|---|---|---|---|---|---|
| | (wt. %) | (wt. %) | (wt. %) | (wt. %) | (wt. %) | (wt. %) | (wt. %) |
| (POG)₅ | 2 | — | — | — | — | — | — |
| PC | — | 76 | — | — | — | — | — |
| FC | — | — | 76 | — | — | — | — |
| PC-CP | — | — | — | 76 | — | — | — |
| PC-2 | — | — | — | — | 76 | — | — |
| FC-2 | — | — | — | — | — | 76 | — |
| PC-CP-2 | — | — | — | — | — | — | 76 |
| PC-CP-Cont | 74 | — | — | — | — | — | — |
| Calcium | 6 | 6 | 6 | 6 | 6 | 6 | 6 |
| (sintered and grained oyster | | | | | | | |
| shell) | | | | | | | |
| Glucosamine hydrochloride | 14 | 14 | 14 | 14 | 14 | 14 | 14 |
| Vitamin C | 4 | 4 | 4 | 4 | 4 | 4 | 4 |

### Example 8

A chewable-type tablet was produced using the aforementioned PC.

Concretely, the following blending ingredients were mixed, and chewable type tablets weighing 0.8 g per tablet were prepared using a tableting machine. This chewable type tablet contained 0.005% of EGDGHLGKPGROGE (SEQ ID NO:1), 0.005% of EKDGHPGKPGROGE (SEQ ID NO:2), 0.005% of (POG)₅, 0.01% of G(POG)₄, 0.05% of (POG)₄, 0.1% of G(POG)₃, 0.5% of (POG)₃, 1% of G(POG)₂, and 2.5% of (POG)₂ in the total of 100%.

PC 50.0 kg

Ascorbic acid 10.0 kg

MICROCALMAG S (produced by SK Foods Co., Ltd.) 4.6 kg

Mabit (produced by Hayashibara Co., Ltd.) 19.0 kg

Crystalline cellulose 10.0 kg

Emulsifying agent 3.2 kg

Aspartame 0.5 kg

Fermented milk powder 1.4 kg

Powder flavor 1.0 kg

Citric acid 0.3 kg

### Example 9

Using the above PC, powder consomme soup (6.0 g per package) to be dissolved in 100 to 140 mL hot water before drinking was prepared by mixing the following blending ingredients. This powder consomme soup contained 0.0035% of EGDGHLGKPGROGE (SEQ ID NO:1), 0.0035% of EKDGHPGKPGROGE (SEQ ID NO:2), 0.0035% of (POG)₅, 0.007% of G(POG)₄, 0.035% of (POG)₄, 0.07% of G(POG)₃, 0.35% of (POG)₃, 0.7% of G(POG)₂, and 1.75% of (POG)₂ in the total of 100%.

PC 35.0 kg

Chicken extract powder 25.0 kg

Sodium chloride 18.0 kg

Glucose 7.7 kg

Calcium lactate 7.0 kg

Sodium glutamate 4.0 kg

Onion extract powder 1.0 kg

HVP 1.0 kg

Beef flavor 0.5 kg

5′-libonucleotide 2 sodium 0.5 kg

White pepper 0.2 kg

Turmeric 0.1 kg

### Example 10

Using the above PC, powder juice (13.0 g per package) to be dissolved in 100 to 150 mL water before drinking was prepared by mixing the following blending ingredients. This powder juice contained 0.004% of EGDGHLGKPGROGE (SEQ ID NO:1), 0.004% of EKDGHPGKPGROGE (SEQ ID NO:2), 0.004% of (POG)₅, 0.008% of G(POG)₄, 0.04% of (POG)₄, 0.08% of G(POG)₃, 0.4% of (POG)₃, 0.8% of G(POG)₂, and 2% of (POG)₂ in the total of 100%.

PC 40.4 kg

Sodium ascorbate 1.2 kg

Erythritol 52.0 kg

Acesulfame K 0.1 kg

Aspartame 0.1 kg

Sodium citrate 0.8 kg

Citric acid (crystal) 4.6 kg

Muscat flavor 0.8 kg

### Example 11

Using the above PC, other blending ingredients were dissolved in purified water according to the following blending ingredients, and adjusted to pH 3.5, B′×9.0%, and then subjected to a heat sterilization treatment at 110° C. for 30 seconds, and cooled to 10° C. and aseptically packed in a paper package, to prepare a soft drink (125 mL per package). This soft drink contained 0.00025% of EGDGHLGKPGROGE (SEQ ID NO:1), 0.00025% of EKDGHPGKPGROGE (SEQ ID NO:2), 0.00025% of (POG)₅, 0.0005% of G(POG)₄, 0.0025% of (POG)₄, 0.005% of G(POG)₃, 0.025% of (POG)₃, 0.05% of G(POG)₂, and 0.125% of (POG)₂ in the total of 100%.

PC 2.5 kg

Vitamin mix DN (produced by BASF Japan) 0.1 kg

Erythritol 5.5 kg

Acesulfame K 0.015 kg

Aspartame 0.005 kg

Citric acid about 0.6 kg

Fruit mix flavor 0.16 L

Lychee flavor 0.04 L

Purified water balance (for making up for the total of 100.0 kg)

### Example 12

First, among the following blending ingredients, the PC and gelatin were immersed with purified water (B) and allowed to swell for 30 minutes, and then they are completely dissolved by heating to 80° C. for 30 minutes, to prepare a gelatin solution. Then, of the following blending ingredients, milk oligosaccharide, powder malt reducing sugar, erythritol, and indigestible dextrin were dissolved in purified water (A), and boiled down, and then thereto was added Aspartame, the aforementioned gelatin solution, citric acid (crystal) dissolved in advance in part of purified water (A), peppermint flavor, mint flavor, lemon flavor and a safflower yellow pigment, and prepared in B′×79 to 81%, and then defoamed, and packed in a starch mold and dried at room temperature for 24 hours, to prepare gummy jelly (4 g per piece). This gummy jelly contained 0.0005% of EGDGHLGKPGROGE (SEQ ID NO:1), 0.0005% of EKDGHPGKPGROGE (SEQ ID NO:2), 0.0005% of (POG)₅, 0.001% of G(POG)₄, 0.005% of (POG)₄, 0.01% of G(POG)₃, 0.05% of (POG)₃, 0.1% of G(POG)₂, 0.25% of (POG)₂ in the total of 100%.

PC 5.0 kg

Milk oligosaccharide 41.0 kg

Powder malt reducing sugar 31.0 kg

Erythritol 5.0 kg

Indigestible dextrin 5.0 kg

Aspartame 0.05 kg

Gelatin (APH250, produced by Nitta Gelatin) 7.0 kg

Citric acid (crystal) 1.2 kg

Peppermint flavor 0.6 L

Mint flavor 0.2 L

Lemon flavor 0.7 L

Safflower yellow pigment appropriate amount

Purified water (A) 20.0 L

Purified water (B) 18.0 L

### Examples 13 to 17

Various disease inhibiting agents were obtained in similar manner to those in Examples 8 to 12 except that PC-2 was used in place of PC.

### Example 18

By solubilizing (POG)₅ of Example 1 in sterilized saline in a concentration of 2.5 mM, a disease inhibiting agent according to Example 18 usable for injection into a diseased site was obtained.

### Examples 19 to 27, Comparative Examples 1 to 3

### Preparation of disease inhibiting agents was conducted according to the paper “Takeshita F, et al. Proc. Natl. Acad. Sci. USA, 2005; 102: 12177-12182”, and tests thereof were conducted in the following manner.

A bone metastasis model was prepared by administering human prostate cancer cell strain PC-3M (PC-3M-lu) that expresses luciferase from the left ventricle of a nude mouse. Then, GL3siRNA that specifically inhibits luciferase was mixed with each synthetic peptide (10 μM) or a conventionally known general DDS carrier and allowed to form a complex, and systemically administered from the tail vein. The mouse was evaluated by IVIS (Real-time in vivo imaging system)(manufactured by Xenogen: Sumisho Bioscience) which measures the amount of luminescence of luciferase in a bone metastatic focus by analyzing in vivo imaging.

The result is shown in Table 11.

TABLE 11

| | | | Luciferase expression |
|---|---|---|---|
| | | | ratio in bone after |
| | | | 28 days from |
| | | | administration (%) |
| Control (only siRNA) | | | 97 ± 1.8 |
| Example | 19 | (POG)₅ | 20 ± 0.8** |
| (synthetic | 20 | EGDGHLGKPGROGE | 16 ± 0.2** |
| peptide) | | (SEQ ID NO: 1) | |
| | 21 | EKDGHPGKPGROGE | 16 ± 0.3** |
| | | (SEQ ID NO: 2) | |
| | 22 | G(POG)₄ | 25 ± 0.7** |
| | 23 | (POG)₄ | 19 ± 0.3** |
| | 24 | G(POG)₃ | 25 ± 0.9** |
| | 25 | (POG)₃ | 21 ± 0.4** |
| | 26 | G(POG)₂ | 27 ± 0.8** |
| | 27 | (POG)₂ | 22 ± 0.6** |
| Comparative | 1 | PVA | 68 ± 2.1* |
| Example | 2 | PEG | 72 ± 1.9* |
| (conventional | 3 | PLA | 59 ± 2.7* |
| DDS carrier) | | | |

(Test number: n = 3)
Note)
**Statistically significant difference in comparison with control (p < 0.01)
*Statistically significant difference in comparison with control (p < 0.05)
Note)
PVA: Polyvinylalcohol (average degree of polymerization about 1500, produced by Wako Pure Chemical Industries)
PEG: Polyethylene glycol (average molecular weight 1500, produced by Wako Pure Chemical Industries)
PLA: Polylactic acid (molecular weight 1600 to 2400, produced by Wako Pure Chemical Industries)

Table 11 reveals that when the peptide molecule having a specific structure of the present invention is used, the luciferase expression ratio is lower and bone metastasis is inhibited and that transfer of siRNA to the target effectively functions in comparison with the case where only siRNA (control) is used or the case where the conventional general DDS carrier is used.

### Examples 28 to 36, Comparative Examples 4 to 6

A bone metastasis nude mouse was intragastrically administered with 0.1 g of each synthetic peptide solubilized in 0.5 mL of distilled water, and after 30 minutes from administration, GL3siRNA that specifically inhibits luciferase was systemically administered from the tail vein of the mouse. For this mouse, evaluation was made in a similar manner to Examples 19 to 27.

The result is shown in Table 12.

TABLE 12

| | | | Luciferase expression |
|---|---|---|---|
| | | | ratio in bone after |
| | | | 28 days from |
| | | | administration (%) |
| Control (only siRNA) | | | 97 ± 2.0 |
| Example | 28 | (POG)₅ | 32 ± 2.4** |
| (synthetic | 29 | EGDGHLGKPGROGE | 26 ± 1.1** |
| peptide) | | (SEQ ID NO: 1) | |
| | 30 | EKDGHPGKPGROGE | 27 ± 0.9** |
| | | (SEQ ID NO: 2) | |
| | 31 | G(POG)₄ | 35 ± 1.9** |
| | 32 | (POG)₄ | 30 ± 1.2** |
| | 33 | G(POG)₃ | 33 ± 1.2** |
| | 34 | (POG)₃ | 27 ± 0.8** |
| | 35 | G(POG)₂ | 29 ± 1.1** |
| | 36 | (POG)₂ | 26 ± 0.9** |
| Comparative | 4 | PVA | 96 ± 2.3 |
| Example | 5 | PEG | 95 ± 1.9 |
| (conventional | 6 | PLA | 93 ± 1.7 |
| DDS carrier) | | | |

(Test number: n = 3)
Note)
**Statistically significant difference in comparison with control (p < 0.01)
Note)
PVA: Polyvinylalcohol (average degree of polymerization about 1500, produced by Wako Pure Chemical Industries)
PEG: Polyethylene glycol (average molecular weight 1500, produced by Wako Pure Chemical Industries)
PLA: Polylactic acid (molecular weight 1600 to 2400, produced by Wako Pure Chemical Industries)

Table 12 reveals that the peptide molecule having a specific structure of the present invention effectively functions as a delivery carrier of siRNA to the target even by co-administration.

### INDUSTRIAL APPLICABILITY

The disease inhibiting agent according to the present invention may be preferably used, for example, as an osteoporosis inhibiting agent, an osteoarthritis inhibiting agent, and a pressure ulcer inhibiting agent, and further as a complex of a nucleic acid compound and a peptide molecule.

1. Glu-Gly-Asp-Gly-His-Leu-Gly-Lys-Pro-Gly-Arg-Hyp-Gly-Glu, Glu-Lys-Asp-Gly-His-Pro-Gly-Lys-Pro-Gly-Arg-Hyp-Gly-Glu, Gly-(Pro-Hyp-Gly)₄ (SEQ ID NO:3), (Pro-Hyp-Gly)₃ (SEQ ID NO:4), Gly-(Pro-Hyp-Gly)₂ (SEQ ID NO:5), (Pro-Hyp-Gly)₂ (SEQ ID NO:6) or (Pro-Hyp-Gly)₄ (SEQ ID NO:7), or a pharmaceutically acceptable salt thereof, or a mixture thereof.

2. A method for inhibiting a disease, comprising administering to a patient in need thereof at least one peptide molecule selected from the group consisting of Glu-Gly-Asp-Gly-His-Leu-Gly-Lys-Pro-Gly-Arg-Hyp-Gly-Glu, Glu-Lys-Asp-Gly-His-Pro-Gly-Lys-Pro-Gly-Arg-Hyp-Gly-Glu, Gly-(Pro-Hyp-Gly)₄ (SEQ ID NO:3), (Pro-Hyp-Gly)₃ (SEQ ID NO:4), Gly-(Pro-Hyp-Gly)₂ (SEQ ID NO:5), (Pro-Hyp-Gly)₂ (SEQ ID NO:6), (Pro-Hyp-Gly)₄ (SEQ ID NO:7), (Pro-Hyp-Gly)₅ (SEQ ID NO:8) and Gly-(Pro-Hyp-Gly)₃ (SEQ ID NO:9), and pharmaceutically acceptable salts thereof.

3. The method according to claim 2, comprising administering to a patient in need thereof at least one peptide molecule selected from the group consisting of Glu-Gly-Asp-Gly-His-Leu-Gly-Lys-Pro-Gly-Arg-Hyp-Gly-Glu, Glu-Lys-Asp-Gly-His-Pro-Gly-Lys-Pro-Gly-Arg-Hyp-Gly-Glu, Gly-(Pro-Hyp-Gly)₄ (SEQ ID NO:3), (Pro-Hyp-Gly)₃ (SEQ ID NO:4), Gly-(Pro-Hyp-Gly)₂ (SEQ ID NO:5), (Pro-Hyp-Gly)₂ (SEQ ID NO:6), (Pro-Hyp-Gly)₄ (SEQ ID NO:7) and Gly-(Pro-Hyp-Gly)₃ (SEQ ID NO:9), and pharmaceutically acceptable salts thereof as an active ingredient.

4. The method according to claim 3, wherein the method is for inhibiting osteoarthritis, osteoporosis or pressure ulcer.

5. The method according to claim 2, comprising administering to a patient in need thereof at least one peptide molecule selected from the group consisting of Glu-Gly-Asp-Gly-His-Leu-Gly-Lys-Pro-Gly-Arg-Hyp-Gly-Glu, Glu-Lys-Asp-Gly-His-Pro-Gly-Lys-Pro-Gly-Arg-Hyp-Gly-Glu, Gly-(Pro-Hyp-Gly)₄ (SEQ ID NO:3), (Pro-Hyp-Gly)₃ (SEQ ID NO:4), Gly-(Pro-Hyp-Gly)₂ (SEQ ID NO:5), (Pro-Hyp-Gly)₂ (SEQ ID NO:6), (Pro-Hyp-Gly)₄ (SEQ ID NO:7), (Pro-Hyp-Gly)₅ (SEQ ID NO:8) and Gly-(Pro-Hyp-Gly)₃ (SEQ ID NO:9), and pharmaceutically acceptable salts thereof as a carrier component.

6. The method according to claim 5, wherein said at least one peptide molecule forms an electrostatic complex with a nucleic acid compound as an active ingredient.

7. The method according to claim 6, wherein said at least one peptide molecule is a delivery agent for a nucleic acid compound that is a drug for inhibiting bone metastasis.

8. The method according to claim 2, adapted for oral administration.

9. The method according to claim 6, comprising administering to a patient in need thereof said at least one peptide molecule orally, and said nucleic acid compound topically.


SEQUENCE LISTING (9 sequences)

SEQ ID NO: 1 - protein, 14 aa, Sus scrofa
  misc_feature (12)..(12): 4Hyp
EGDGHLGKPGRXGE

SEQ ID NO: 2 - protein, 14 aa, Sus scrofa
  MISC_FEATURE (12)..(12): 4Hyp
EKDGHPGKPGRXGE

SEQ ID NO: 3 - protein, 13 aa, Sus scrofa
  MISC_FEATURE (3)..(3): 4Hyp
GPXGPXGPXGPXG

SEQ ID NO: 4 - protein, 9 aa, Sus scrofa
  MISC_FEATURE (2)..(2): 4Hyp
PXGPXGPXG

SEQ ID NO: 5 - protein, 7 aa, Sus scrofa
  MISC_FEATURE (3)..(3): 4Hyp
GPXGPXG

SEQ ID NO: 6 - protein, 6 aa, Sus scrofa
  MISC_FEATURE (2)..(2): 4Hyp
PXGPXG

SEQ ID NO: 7 - protein, 12 aa, Sus scrofa
  MISC_FEATURE (2)..(2): 4Hyp
PXGPXGPXGPXG

SEQ ID NO: 8 - protein, 15 aa, Sus scrofa
  MISC_FEATURE (2)..(2): 4Hyp
PXGPXGPXGPXGPXG

SEQ ID NO: 9 - protein, 10 aa, Sus scrofa
  MISC_FEATURE (3)..(3): 4Hyp
GPXGPXGPXG